(19) United States
(12) Patent Application Publication
(10) Pub. No.: US 2023/0406322 A1
(43) Pub. Date: Dec. 21, 2023

(54) AWAKENING LEVEL ESTIMATION DEVICE, AWAKENING LEVEL ESTIMATION METHOD, AWAKENING LEVEL LEARNING DEVICE, AND AWAKENING LEVEL LEARNING METHOD

(71) Applicant: Mitsubishi Electric Corporation, Tokyo (JP)

(72) Inventors: Atsushi MATSUMOTO, Tokyo (JP); Shintaro WATANABE, Tokyo (JP); Yumiko OKAMOTO, Tokyo (JP); Genta YOSHIMURA, Tokyo (JP)

(73) Assignee: Mitsubishi Electric Corporation, Tokyo (JP)

(21) Appl. No.: 18/031,072

(22) PCT Filed: Dec. 28, 2020
(86) PCT No.: PCT/JP2020/049059

§ 371 Date: Apr. 10, 2023

(51) Int. Cl. B60W 40/08; G06N 20/00

(52) U.S. Cl. CPC B60W 40/08; B60W 2540/229; B60W 2540/043; G06N 20/00

(57) ABSTRACT

An awakening level estimation device includes: processing circuitry configured to acquire two or more types of occupant state information different from each other, the occupant state information indicating a current state value of an occupant; acquire occupant basic state information indicating a basic state value of the occupant when the occupant is in an awakening state; acquire difference information indicating a difference between the current state value and the basic state value, estimate an awakening level of the occupant on the basis of the two or more types of difference information, and input the two or more types of difference information to a learned model corresponding to a leaning result by machine learning, and generating and outputting awakening level information indicating an awakening level on the basis of an estimation result output from the leaned model.

### TECHNICAL FIELD

The present disclosure relates to an awakening level estimation device, an awakening level estimation method, an awakening level learning device, and an awakening level learning method.

### BACKGROUND ART

There is a technique of determining whether or not an occupant of a vehicle is awake (hereinafter, referred to as “awakening state”) using information indicating a state of the occupant (hereinafter, referred to as “occupant state information”).

For example, Patent Literature 1 discloses a technique of specifying, as an awakening section, a section in which an awakening level of an occupant (hereinafter, also referred to as “subject”) has moved in an awakening direction from a section immediately after the subject gets in a vehicle using a heart rate of the subject, calculating a statistical value of the awakening level included in the specified awakening section, and setting, for each subject, a threshold for determining an awakening state of the subject on the basis of the calculated statistical value, in which whether or not the subject is in an awakening state is determined by determining whether or not the awakening level of the subject is less than the threshold.

### CITATION LIST

### Patent Literature

Patent Literature 1: JP 2016-165349 A

### SUMMARY OF INVENTION

### Technical Problem

As described above, the technique disclosed in Patent Literature 1 (hereinafter, referred to as “related art”) uses only one type of occupant state information (heart rate) as a parameter for determining whether or not an occupant is in an awakening state.

Related art has a problem that whether or not an occupant is in an awakening state cannot be determined using two or more types of occupant state information as parameters.

The present disclosure has been made in order to solve the above problem, and an object of the present disclosure is to provide an awakening level estimation device capable of estimating an awakening level of an occupant using two or more types of occupant state information.

### Solution to Problem

An awakening level estimation device according to the present disclosure includes: an occupant state acquiring unit that acquires two or more types of occupant state information different from each other, the occupant state information indicating a current state value which is a state value of an occupant of a vehicle; an occupant basic state acquiring unit that acquires occupant basic state information corresponding to each of the two or more types of occupant state information acquired by the occupant state acquiring unit and indicating a basic state value which is the state vale of the occupant when the occupant is in an awakening state; a difference acquiring unit that acquires difference information indicating a difference between the current state value indicated by the occupant state information acquired by the occupant state acquiring unit and the basic state value indicated by the occupant basic state information acquired by the occupant basic state acquiring unit and corresponding to the occupant state information, the difference acquiring unit acquiring two or more types of the difference information corresponding to the two or more types of occupant state information acquired by the occupant state acquiring unit; and an awakening level estimating unit that estimates an awakening level of the occupant on the basis of the two or more types of difference information acquired by the difference acquiring unit, the awakening level estimating unit inputting the two or more types of difference information to a learned model corresponding to a leaning result by machine learning, and generating and outputting awakening level information indicating the awakening level on the basis of an estimation result output from the leaned model.

### Advantageous Effects of Invention

According to the present disclosure, it is possible to estimate an awakening level of an occupant using two or more types of occupant state information.

### BRIEF DESCRIPTION OF DRAWINGS

FIG. 1 is a block diagram illustrating an example of a configuration of a main part of an awakening level estimation system to which an awakening level estimation device according to a first embodiment is applied.

FIG. 2 is a block diagram illustrating an example of a configuration of a main part of the awakening level estimation device according to the first embodiment.

FIG. 3 is a block diagram illustrating an example of a configuration of a main part of an occupant state acquiring unit included in the awakening level estimation device according to the first embodiment.

FIGS. 4A and 4B are diagrams illustrating an example of a hardware configuration of the main part of the awakening level estimation device according to the first embodiment.

FIG. 5 is a flowchart for explaining an example of processing of the awakening level estimation device according to the first embodiment.

FIG. 6 is a flowchart for explaining another example of the processing of the awakening level estimation device according to the first embodiment.

FIG. 7 is a block diagram illustrating an example of a configuration of a main part of an awakening level learning system to which an awakening level learning device according to the first embodiment is applied.

FIG. 8 is a block diagram illustrating an example of a configuration of a main part of the awakening level learning device according to the first embodiment.

FIGS. 9A and 9B are diagrams illustrating an example of a hardware configuration of the main part of the awakening level learning device according to the first embodiment.

FIG. 10 is a flowchart for explaining an example of processing of the awakening level learning device according to the first embodiment.

FIG. 11 is a flowchart for explaining another example of the processing of the awakening level learning device according to the first embodiment.

FIG. 12 is a block diagram illustrating an example of a configuration of a main part of an awakening level estimation system to which an awakening level estimation device according to a second embodiment is applied.

FIG. 13 is a block diagram illustrating an example of a configuration of a main part of the awakening level estimation device according to the second embodiment.

FIG. 14 is a flowchart for explaining an example of processing of the awakening level estimation device according to the second embodiment.

### DESCRIPTION OF EMBODIMENTS

Hereinafter, embodiments of the present disclosure will be described in detail with reference to the drawings.

### First Embodiment

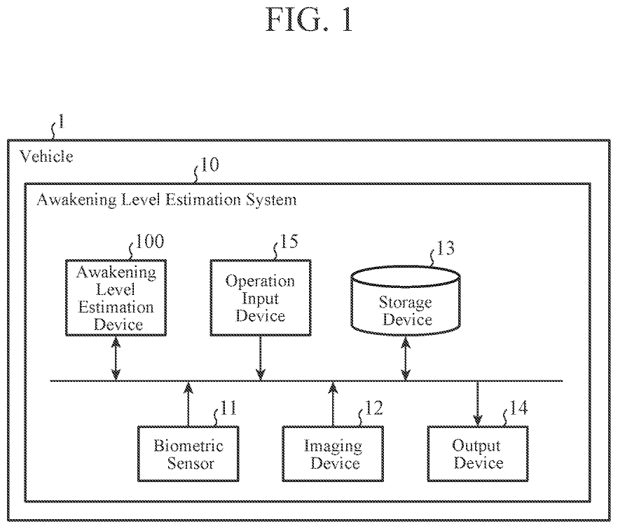

An awakening level estimation device 100 according to a first embodiment will be described with reference to FIGS. 1 to 6.

With reference to FIG. 1, a configuration of a main part of an awakening level estimation system 10 to which the awakening level estimation device 100 according to the first embodiment is applied will be described.

FIG. 1 is a block diagram illustrating an example of the configuration of the main part of the awakening level estimation system 10 to which the awakening level estimation device 100 according to the first embodiment is applied.

The awakening level estimation system 10 is mounted on a vehicle 1.

The awakening level estimation system 10 includes a biometric sensor 11, an imaging device 12, a storage device 13, an output device 14, an operation input device and the awakening level estimation device 100.

The biometric sensor 11 detects a state related to a living body of an occupant in the vehicle 1, such as expiration in respiration or inspiration, heartbeat, or a body temperature of the occupant, converts the detected state related to the living body of the occupant into a sensor signal that is an electric signal, and outputs the sensor signal. The biometric sensor 11 is constituted by a Doppler sensor, a vibration sensor, a thermo-sensor, or the like, and is disposed on a seat, a seat belt, a steering wheel, or the like included in the vehicle 1. A heart rate per unit time such as one minute, a heart rate variation value in a predetermined period such as ten minutes, a respiration rate per unit time such as one minute, a respiration cycle in a predetermined period such as ten minutes, a body temperature, and the like can be calculated on the basis of the sensor signal output from the biometric sensor 11.

Hereinafter, the biometric sensor 11 will be described as a heartbeat sensor that detects vibration caused by heartbeat, converts the detected vibration into a sensor signal, and outputs the sensor signal.

Note that the biometric sensor 11 included in the awakening level estimation system 10 is not limited to one that detects vibration due to heartbeat, and the number of biometric sensors 11 included in the awakening level estimation system 10 is not limited to one. For example, the awakening level estimation system 10 may include two or more biometric sensors 11 whose targets to be detected are different from each other.

The imaging device 12 images a face, an upper body, or the like of an occupant and outputs an image obtained by the imaging as image information. The imaging device 12 is constituted by a digital camera, a digital video camera, or the like, and is disposed, for example, in a front portion in the vehicle interior of the vehicle 1.

Only one imaging device 12 may be disposed in the vehicle 1 and may be capable of imaging the entire vehicle interior from the front portion in the vehicle interior, or the imaging devices 12 may be disposed corresponding to a plurality of seats included in the vehicle 1, respectively, and may image occupants seated on the seats.

The storage device 13 stores information necessary for the awakening level estimation device 100 to execute predetermined processing determined in advance. The awakening level estimation device 100 can acquire information 13 by reading out the information stored in the storage device.

The output device 14 is a display device such as a display that displays and outputs a display image, a sound device such as a speaker that outputs sound, a vibration device including a piezoelectric element or the like that converts an electric signal into vibration and outputs the vibration, or the like. The output device 14 performs display output, sound output, vibration output, or the like on the basis of a control signal output from the awakening level estimation device 100.

The output device 14 may be an air conditioner that adjusts a temperature in a vehicle interior or an electronic control unit (ECU) that controls a prime mover or a brake that causes the vehicle 1 to travel or stop.

In a case where the output device 14 is an air conditioner, the output device 14 controls a temperature in a vehicle interior on the basis of the control signal output from the awakening level estimation device 100.

In a case where the output device 14 is an electronic control unit, the output device 14 controls a prime mover, a brake, or the like of the vehicle 1 on the basis of the control signal output from the awakening level estimation device 100, and causes the vehicle 1 to stop, for example.

The operation input device 15 receives an operation of an occupant and outputs an operation signal based on the operation. For example, the occupant performs an operation input by tapping the operation input device 15 constituted by a touch panel. The occupant may perform the operation input by performing a voice input to the operation input device 15 constituted by a voice recognition device or the like.

The awakening level estimation device 100 receives the sensor signal output from the biometric sensor 11 and the image information output from the imaging device 12, and estimates the awakening level of the occupant on the basis of the sensor signal and the image information. The awakening level estimation device 100 outputs awakening level information indicating the estimated awakening level or a control signal based on the estimated awakening level.

The awakening level estimation device 100 may receive only either one of the sensor signal output from the biometric sensor 11 and the image information output from the imaging device 12. In a case where the awakening level estimation device 100 receives only the sensor signal output from the biometric sensor 11, the awakening level estimation system 10 includes two or more biometric sensors 11 whose targets to be detected are different from each other, and the awakening level estimation device 100 receives sensor signals output from the two or more biometric sensors 11 whose targets to be detected are different from each other, and estimates the awakening level of the occupant on the basis of the sensor signals.

Hereinafter, description will be given on the assumption that the awakening level estimation device 100 receives the sensor signal output from the biometric sensor 11 constituted by a heartbeat sensor and the image information output from the imaging device 12, estimates the awakening level of the occupant on the basis of the sensor signal and the image information, and outputs a control signal based on the estimated awakening level.

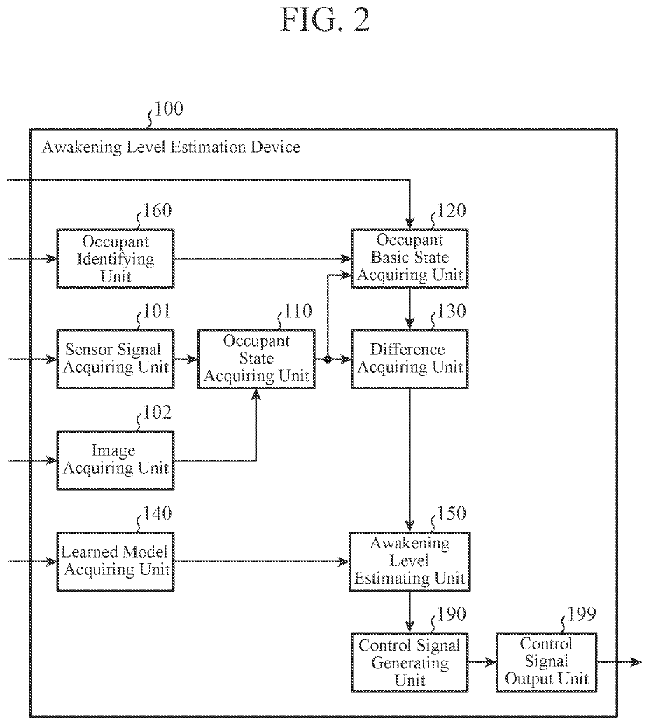

A configuration of a main part of the awakening level estimation device 100 according to the first embodiment will be described with reference to FIG. 2.

FIG. 2 is a block diagram illustrating an example of the configuration of the main part of the awakening level estimation device 100 according to the first embodiment.

The awakening level estimation device 100 includes a sensor signal acquiring unit 101, an image acquiring unit 102, an occupant state acquiring unit 110, an occupant basic state acquiring unit 120, a difference acquiring unit 130, a learned model acquiring unit 140, an awakening level estimating unit 150, a control signal generating unit 190, and a control signal output unit 199.

The awakening level estimation device 100 may include an occupant identifying unit 160 in addition to the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the control signal generating unit 190, and the control signal output unit 199.

Hereinafter, the awakening level estimation device 100 will be described as including the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the control signal generating unit 190, and the control signal output unit 199.

The sensor signal acquiring unit 101 acquires a sensor signal output from the biometric sensor 11 that outputs a sensor signal obtained by detecting a state related to a living body of an occupant.

The image acquiring unit 102 acquires image information output from the imaging device 12 that outputs image information indicating an image obtained by imaging the occupant.

The occupant identifying unit 160 identifies an occupant in the vehicle 1. The occupant identifying unit 160 acquires personal identification information indicating a person identified by the occupant identifying unit 160.

Specifically, for example, the occupant identifying unit 160 identifies an occupant in the vehicle 1 on the basis of an operation signal output from the operation input device 15.

For example, an occupant inputs information capable of identifying the occupant by operating the operation input device 15. The operation input device 15 outputs an operation signal based on the operation to the awakening level estimation device 100. The occupant identifying unit 160 identifies an occupant in the vehicle 1 on the basis of the operation signal output from the operation input device 15.

A method by which the occupant identifying unit 160 identifies an occupant in the vehicle 1 is not limited to the method based on the operation signal output from the operation input device 15 such as a touch panel.

For example, the occupant identifying unit 160 may receive a signal output from a fingerprint sensor or a voice input device or the like such as a microphone (not illustrated in FIG. 1), and may identify an occupant in the vehicle 1 on the basis of fingerprint authentication, voiceprint authentication, a voice input signal, or the like.

In addition, for example, the occupant identifying unit 160 may analyze an image indicated by image information acquired by the image acquiring unit 102 by a known image analysis technique, and may identify an occupant in the vehicle 1 by authenticating the face of the occupant.

The occupant state acquiring unit 110 acquires two or more types of occupant state information different from each other, the occupant state information indicating a state value (hereinafter, referred to as “current state value”) of the occupant in the vehicle 1.

Specifically, for example, the occupant state acquiring unit 110 acquires first occupant state information (hereinafter, referred to as “first occupant state information”) indicating a first state value (hereinafter, referred to as “first current state value”) of the occupant in the vehicle 1 on the basis of the sensor signal acquired by the sensor signal acquiring unit 101. In addition, the occupant state acquiring unit 110 acquires second occupant state information (hereinafter, referred to as “second occupant state information”) indicating a second state value (hereinafter, referred to as “second current state value”) of the occupant in the vehicle 1 on the basis of the image information acquired by the image acquiring unit 102.

The occupant state acquiring unit 110 only needs to acquire two or more types of occupant state information different from each other, and is not limited to one that acquires the first and second occupant state information. That is, in a case where the occupant state acquiring unit 110 acquires N (N is an integer of 2 or more) types of occupant state information different from each other, the occupant state acquiring unit 110 acquires n-th occupant state information (hereinafter, referred to as “n-th occupant state information”) indicating each of n-th (n indicates all integers of 1 to N) state values (hereinafter, referred to as “n-th current state value”) of the occupant in the vehicle 1 on the basis of the sensor signal acquired by the sensor signal acquiring unit 101 or the image information acquired by the image acquiring unit 102.

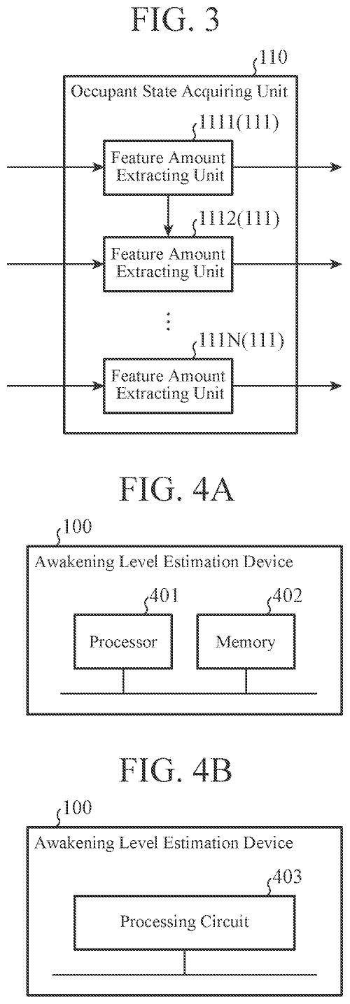

A configuration of a main part of the occupant state acquiring unit 110 according to the first embodiment will be described with reference to FIG. 3.

FIG. 3 is a block diagram illustrating an example of the configuration of the main part of the occupant state acquiring unit 110 included in the awakening level estimation device 100 according to the first embodiment.

The occupant state acquiring unit 110 includes N feature amount extracting units 111 (feature amount extracting unit 1111, 1112, . . . , 111N).

Each of the N feature amount extracting units 111 receives the sensor signal acquired by the sensor signal acquiring unit 101 or the image information acquired by the image acquiring unit 102, and generates and acquires occupant state information on the basis of the sensor signal or the image information. The types of the occupant state information acquired by the N feature amount extracting units 111 are different from each other.

Specifically, for example, in a case where a certain feature amount extracting unit 111 receives a sensor signal output from the biometric sensor 11 constituted by a heartbeat sensor, the feature amount extracting unit 111 calculates a heart rate per unit time of an occupant or a heart rate variation value of the occupant in a predetermined period on the basis of the sensor signal. The feature amount extracting unit 111 acquires the calculated heart rate per unit time of the occupant or the heart rate variation value of the occupant in the predetermined period as occupant state information.

In addition, for example, in a case where a certain feature amount extracting unit 111 receives a sensor signal output from the biometric sensor 11 constituted by a Doppler sensor or the like, the feature amount extracting unit 111 may calculate a respiration rate per unit time of an occupant or a respiration cycle of the occupant in a predetermined period on the basis of the sensor signal, and may acquire the respiration rate or the respiration cycle as occupant state information.

In addition, for example, in a case where a certain feature amount extracting unit 111 receives a sensor signal output from the biometric sensor 11 constituted by a thermo-sensor or the like, the feature amount extracting unit 111 may calculate a body temperature of an occupant on the basis of the sensor signal, and may acquire the body temperature as occupant state information.

In addition, for example, in a case where a certain feature amount extracting unit 111 receives image information acquired by the image acquiring unit 102, the feature amount extracting unit 111 analyzes an image indicated by the image information by a known image analysis technique, and calculates a distance (hereinafter, referred to as “eye opening distance”) from a lower eyelid to an upper eyelid of an occupant. The feature amount extracting unit 111 acquires the calculated eye opening distance of the occupant as occupant state information. The feature amount extracting unit 111 may analyze the image indicated by the image information by a known image analysis technique, may calculate the number of changes in the position of a hand or the direction of a line of sight of the occupant per unit time, the number of blinks per unit time (hereinafter, referred to as “blinking frequency”) of the occupant, or the like, and may acquire the calculated number of changes, the calculated number of blinks, or the like as occupant state information.

Note that, in the above description, the awakening level estimation device 100 includes the sensor signal acquiring unit 101 and the image acquiring unit 102, but the awakening level estimation device 100 may include either one of the sensor signal acquiring unit 101 and the image acquiring unit 102.

For example, in a case where the awakening level estimation device 100 includes the sensor signal acquiring unit 101 but does not include the image acquiring unit 102, the occupant state acquiring unit 110 acquires two or more types of occupant state information different from each other on the basis of one or more sensor signals acquired by the sensor signal acquiring unit 101.

For example, in a case where the awakening level estimation device 100 includes the image acquiring unit 102 but does not include the sensor signal acquiring unit 101, the occupant state acquiring unit 110 acquires two or more types of occupant state information different from each other on the basis of the image information acquired by the image acquiring unit 102.

In the above description, the awakening level estimation device 100 includes the sensor signal acquiring unit 101 and the image acquiring unit 102, and the occupant state acquiring unit 110 includes the N feature amount extracting units 111. However, the sensor signal acquiring unit 101, the image acquiring unit 102, and the N feature amount extracting units 111 included in the occupant state acquiring unit 110 may be included in an external device (not illustrated in FIG. 1) such as an occupant state acquiring device different from the awakening level estimation device 100. In a case where the external device such as the occupant state acquiring device includes the sensor signal acquiring unit 101, the image acquiring unit 102, and the N feature amount extracting units 111 included in the occupant state acquiring unit 110, for example, the occupant state acquiring unit 110 acquires two or more types of occupant state information different from each other by acquiring the n-th occupant state information acquired by the external device from the external device.

The occupant basic state acquiring unit 120 acquires occupant basic state information corresponding to each of the two or more types of occupant state information acquired by the occupant state acquiring unit 110 and indicating a state value (hereinafter, referred to as “basic state value”) of the occupant when the occupant is in an awakening state.

The occupant basic state information corresponding to the occupant state information is occupant basic state information indicating a basic state value corresponding to a current state value such as a heart rate or an eye opening distance indicated by the occupant state information, such as a heart rate or an eye opening distance when the occupant is in an awakening state.

Specifically, the occupant basic state acquiring unit 120 acquires occupant basic state information corresponding to each of pieces of the n-th occupant state information acquired by the occupant state acquiring unit 110. Hereinafter, description will be given by referring to occupant basic state information corresponding to k-th (k is any integer of 1 to N) occupant state information as k-th occupant basic state information.

For example, the occupant basic state acquiring unit 120 acquires, as the k-th occupant basic state information, information indicating a statistical value such as an average value, a median value, or a mode value of current state values indicated by the k-th occupant state information acquired by the occupant state acquiring unit 110 in a period from start of new traveling of the vehicle 1 with a predetermined period such as one hour apart until a predetermined period such as 30 minutes elapses.

When the awakening level estimation device 100 includes the occupant identifying unit 160, for example, the occupant basic state acquiring unit 120 acquires occupant basic state information corresponding to personal identification information acquired by the occupant identifying unit 160 on the basis of the personal identification information.

Specifically, for example, by reading occupant basic state information corresponding to personal identification information acquired by the occupant identifying unit 160 from the storage device 13, the occupant basic state acquiring unit 120 acquires the occupant basic state information corresponding to the personal identification information.

In this case, the occupant basic state information acquired by the occupant basic state acquiring unit 120 is, for example, information indicating a statistical value of a current state value indicated by occupant state information of the occupant indicated by the personal identification information, acquired by the occupant state acquiring unit 110 in a period in which the occupant is in an awakening state in a period in which the occupant was in the vehicle 1 in the past.

The occupant basic state information acquired by the occupant basic state acquiring unit 120 is preferably, for example, information indicating a statistical value of a current state value indicated by occupant state information acquired by the occupant state acquiring unit 110 in a period excluding a period of a special travel state which is a predetermined travel state in a travel state of the vehicle 1.

The period of the special travel state means, for example, a period in which the vehicle 1 is stopped (hereinafter, referred to as a “stop period”), a period from start to end of lane change of the vehicle 1 (hereinafter, referred to as “lane change period”), a period from start to end of right/left turning of the vehicle 1 (hereinafter, referred to as “right/left turning period”), a period in which an occupant is estimated to be having a conversation (hereinafter, referred to as “conversation period”), a period in which the vehicle 1 is traveling in a congested road section (hereinafter, referred to as “congestion period”), a period in which the vehicle 1 is traveling in a road section in which the vehicle 1 has not traveled (hereinafter, referred to as “initial view travel period”), a period in which the vehicle 1 is traveling in a road section narrower than a predetermined road width such as less than 4 m (meters) (hereinafter referred to as “narrow travel period”), a period in which the vehicle 1 is traveling in a predetermined time period such as a midnight time period (hereinafter, referred to as “predetermined time period travel period”), a period in which the vehicle 1 is traveling in predetermined weather such as rainy weather (hereinafter, referred to as “travel period in predetermined weather”), or a period in which the number of times a predetermined driving operation has been performed per unit time such as one minute exceeds a predetermined number of times such as five times (hereinafter, referred to as “complicated driving period”).

The predetermined driving operation in the complicated driving period is, for example, a steering wheel operation, an accelerator operation, a brake operation, or a horn operation.

The difference acquiring unit 130 acquires difference information indicating a difference between the current state value indicated by the occupant state information acquired by the occupant state acquiring unit 110 and a basic state value indicated by the occupant basic state information acquired by the occupant basic state acquiring unit 120 and corresponding to the occupant state information.

Specifically, the difference information acquired by the difference acquiring unit 130 is two or more types of difference information corresponding to two or more types of occupant state information acquired by the occupant state acquiring unit 110.

More specifically, the difference acquiring unit 130 acquires N types of difference information (hereinafter, referred to as “n-th difference information”) by calculating a difference between a current state value indicated by the k-th occupant state information acquired by the occupant state acquiring unit 110 and a basic state value indicated by the k-th occupant basic state information acquired by the occupant basic state acquiring unit 120, and acquiring difference information (hereinafter, referred to as “k-th difference information”) indicating the calculated difference.

The learned model acquiring unit 140 acquires learned model information indicating a learned model corresponding to a learning result by machine learning.

Specifically, for example, the learned model acquiring unit 140 acquires the learned model information by reading the learned model information from the storage device 13 in which the learned model information is stored in advance.

A method for generating the learned model indicated by the learned model information will be described later.

The awakening level estimating unit 150 estimates the awakening level of an occupant on the basis of the two or more types of difference information acquired by the difference acquiring unit 130.

Specifically, the awakening level estimating unit 150 inputs the two or more types of difference information acquired by the difference acquiring unit 130 to the learned model indicated by the learned model information acquired by the learned model acquiring unit 140. The awakening level estimating unit 150 generates and outputs awakening level information indicating an awakening level on the basis of an estimation result output from the learned model.

For example, the learned model indicated by the learned model information acquired by the learned model acquiring unit 140 outputs a numerical value indicating the awakening level of the occupant in a predetermined format such as a percentage as the estimation result. The learned model may output, as the estimation result, a reliability of the numerical value in a predetermined format such as a percentage in addition to the numerical value indicating the awakening level of the occupant.

For example, the awakening level estimating unit 150 converts a numerical value indicating the awakening level output from the learned model into a predetermined level such as any one of five levels on the basis of the estimation result output from the learned model indicated by the learned model information acquired by the learned model acquiring unit 140, and outputs the converted information as the awakening level information.

The awakening level estimating unit 150 may output a numerical value indicating the awakening level that is the estimation result output from the learned model indicated by the learned model information acquired by the learned model acquiring unit 140 as the awakening level information without converting the numerical value into a level or the like.

With the above configuration, the awakening level estimation device 100 can estimate the awakening level of the occupant using the two or more types of occupant state information.

The control signal generating unit 190 generates a control signal on the basis of the awakening level information output from the awakening level estimating unit 150.

Specifically, for example, the control signal generating unit 190 determines whether or not the awakening level indicated by the awakening level information is less than a predetermined threshold (hereinafter, referred to as “awakening threshold”). In a case where the control signal generating unit 190 determines that the awakening level indicated by the awakening level information is less than the awakening threshold, the control signal generating unit 190 generates a control signal for improving the awakening level of the occupant, a control signal for stopping traveling of the vehicle 1, or the like.

The control signal output unit 199 outputs the control signal generated by the control signal generating unit 190 to the output device 14.

The control signal output unit 199 causes the output device 14 to perform control based on the control signal by outputting the control signal to the output device 14.

With the above configuration, the awakening level estimation device 100 estimates the awakening level of the occupant using the two or more types of occupant state information, and in a case where it is estimated that the awakening level of the occupant does not reach the predetermined awakening level, the awakening level estimation device 100 can cause the output device 14 to perform control for improving the awakening level of the occupant, control for stopping traveling of the vehicle 1, or the like.

Note that, in the above description, the awakening level estimation device 100 includes the control signal generating unit 190 and the control signal output unit 199, but the control signal generating unit 190 and the control signal output unit 199 may be included in an external device (not illustrated in FIG. 1) such as a control device different from the awakening level estimation device 100. In a case where the external device such as a control device includes the control signal generating unit 190 and the control signal output unit 199, for example, the awakening level estimation device 100 outputs the awakening level information generated by the awakening level estimating unit 150 to the external device, and causes the external device to generate and output a control signal based on the awakening level information output from the awakening level estimation device 100.

A hardware configuration of a main part of the awakening level estimation device 100 according to the first embodiment will be described with reference to FIGS. 4A and 4B.

FIGS. 4A and 4B are diagrams illustrating an example of a hardware configuration of the main part of the awakening level estimation device 100 according to the first embodiment.

As illustrated in FIG. 4A, the awakening level estimation device 100 is constituted by a computer, and the computer includes a processor 401 and a memory 402. The memory 402 stores a program for causing the computer to function as the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the control signal generating unit 190, and the control signal output unit 199. By the processor 401 reading and executing the program stored in the memory 402, the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the control signal generating unit 190, and the control signal output unit 199 are implemented.

In addition, as illustrated in FIG. 4B, the awakening level estimation device 100 may be constituted by a processing circuit 403. In this case, the functions of the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the control signal generating unit 190, and the control signal output unit 199 may be implemented by the processing circuit 403.

Alternatively, the awakening level estimation device 100 may be constituted by the processor 401, the memory 402, and the processing circuit 403 (not illustrated). In this case, some of the functions of the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the control signal generating unit 190, and the control signal output unit 199 may be implemented by the processor 401 and the memory 402, and the remaining functions may be implemented by the processing circuit 403.

The processor 401 uses, for example, a central processing unit (CPU), a graphics processing unit (GPU), a microprocessor, a microcontroller, or a digital signal processor (DSP).

The memory 402 uses, for example, a semiconductor memory or a magnetic disk. More specifically, the memory 402 uses a random access memory (RAM), a read only memory (ROM), a flash memory, an erasable programmable read only memory (EPROM), an electrically erasable programmable read-only memory (EEPROM), a solid state drive (SSD), a hard disk drive (HDD), or the like.

The processing circuit 403 uses, for example, an application specific integrated circuit (ASIC), a programmable logic device (PLD), a field-programmable gate array (FPGA), a system-on-a-chip (SoC), or a system large-scale integration (LSI).

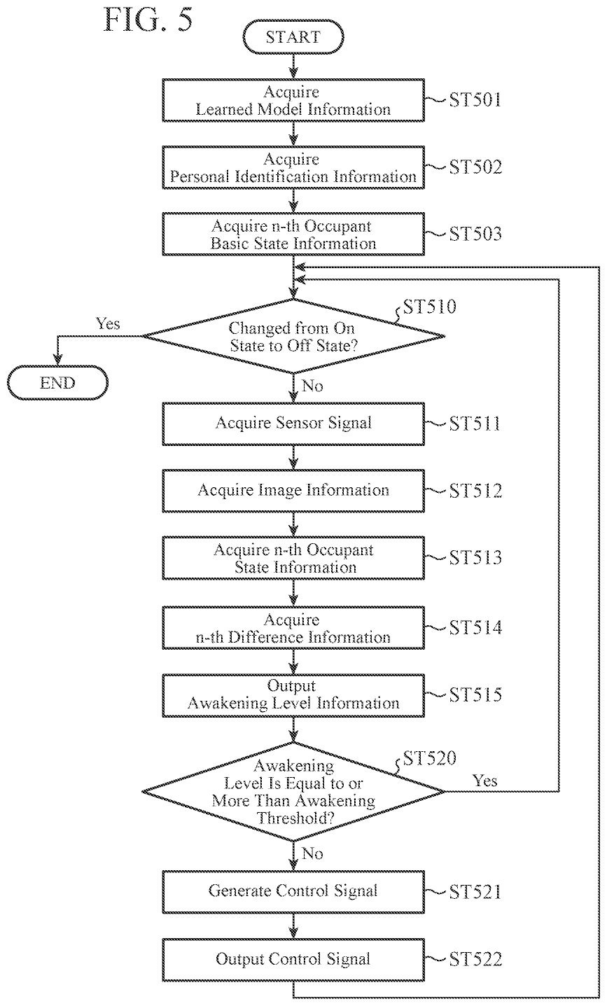

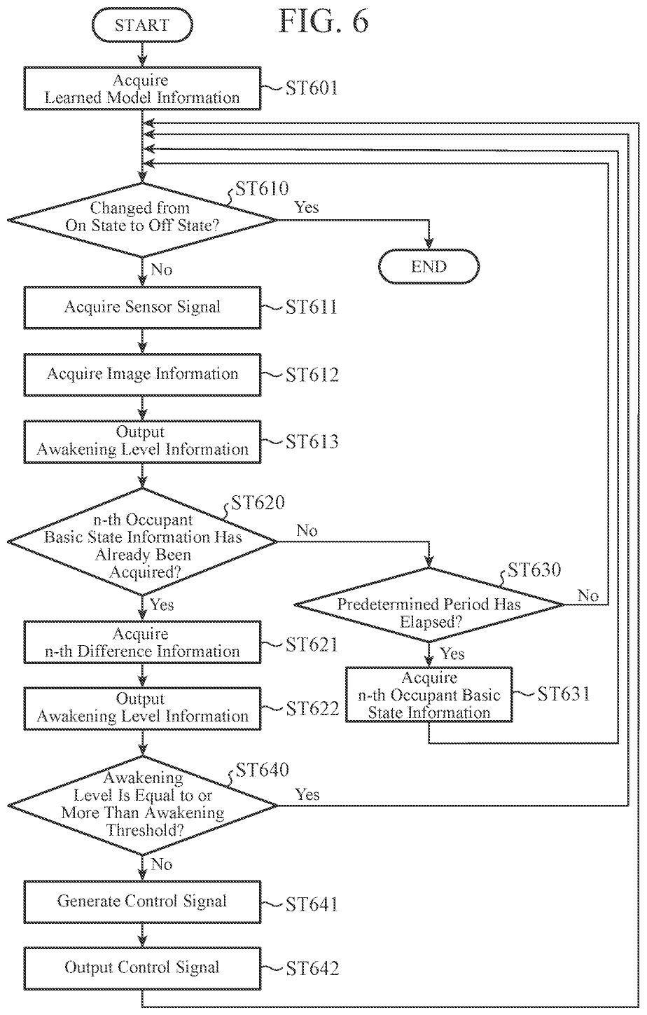

An operation of the awakening level estimation device 100 according to the first embodiment will be described with reference to FIGS. 5 and 6.

FIG. 5 is a flowchart for explaining an example of processing of the awakening level estimation device 100 according to the first embodiment.

Note that, for example, the awakening level estimation device 100 starts processing of the flowchart when an accessory power supply or an ignition power supply changes from an OFF state to an ON state, and ends the processing of the flowchart when the accessory power supply or the ignition power supply changes from the ON state to the OFF state.

In addition, the flowchart illustrated in FIG. 5 illustrates, as an example, processing of the awakening level estimation device 100 in a case where occupant basic state information for each occupant is stored in the storage device 13 in advance.

When the accessory power supply or the ignition power supply changes from the OFF state to the ON state, first, in step ST501, the learned model acquiring unit 140 acquires learned model information.

Next, in step ST502, the occupant identifying unit 160 acquires personal identification information.

Next, in step ST503, the occupant basic state acquiring unit 120 acquires n-th occupant basic state information.

Next, in step ST510, for example, a power supply determining unit (not illustrated in FIG. 2) included in the awakening level estimation device 100 determines whether or not the accessory power supply or the ignition power supply has changed from the ON state to the OFF state.

In step ST510, if the power supply determining unit determines that the accessory power supply or the ignition power supply has changed from the ON state to the OFF state, the awakening level estimation device 100 ends the processing of the flowchart.

In step ST510, if the power supply determining unit determines that the accessory power supply or the ignition power supply has not changed from the ON state to the OFF state, that is, if the power supply determining unit determines that the accessory power supply or the ignition power supply remains in the ON state, the awakening level estimation device 100 executes processing in step ST511 and subsequent steps described below.

In step ST510, if the power supply determining unit determines that the accessory power supply or the ignition power supply has not changed from the ON state to the OFF state, that is, if the power supply determining unit determines that the accessory power supply or the ignition power supply remains in the ON state, first, in step ST511, the sensor signal acquiring unit 101 acquires a sensor signal.

Next, in step ST512, the image acquiring unit 102 acquires image information.

Next, in step ST513, the occupant state acquiring unit 110 acquires n-th occupant state information.

Next, in step ST514, the difference acquiring unit 130 acquires n-th difference information.

Next, in step ST515, the awakening level estimating unit 150 generates and outputs awakening level information.

Next, in step ST520, the control signal generating unit 190 determines whether or not the awakening level indicated by the awakening level information is equal to or more than the awakening threshold.

In step ST520, if the control signal generating unit 190 determines that the awakening level indicated by the awakening level information is equal to or more than the awakening threshold, the awakening level estimation device 100 returns to the processing in step ST510 and executes the processing in step ST510.

In step ST520, if the control signal generating unit 190 determines that the awakening level indicated by the awakening level information is not equal to or more than the awakening threshold, that is, if the control signal generating unit 190 determines that the awakening level indicated by the awakening level information is less than the awakening threshold, in step ST521, the control signal generating unit 190 generates a control signal.

After step ST521, in step ST522, the control signal output unit 199 outputs a control signal.

After step ST522, the awakening level estimation device 100 returns to the processing in step ST510 and executes the processing in step ST510.

In the flowchart illustrated in FIG. 5, the processing from step ST501 to step ST503 may be executed in any order as long as the processing in step ST502 is executed before the processing in step ST503 is executed. In addition, the processing in step ST511 and the processing in step ST512 may be executed in any order.

FIG. 6 is a flowchart for explaining another example of the processing of the awakening level estimation device 100 according to the first embodiment.

Note that, for example, the awakening level estimation device 100 starts processing of the flowchart when an accessory power supply or an ignition power supply changes from an OFF state to an ON state, and ends the processing of the flowchart when the accessory power supply or the ignition power supply changes from the ON state to the OFF state.

The flowchart illustrated in FIG. 6 indicates, as an example, processing of the awakening level estimation device 100 in a case where the occupant basic state acquiring unit 120 acquires, as k-th occupant basic state information, information indicating a statistical value of a current state value indicated by the k-th occupant state information acquired by the occupant state acquiring unit 110 in a period from start of new traveling of the vehicle 1 until a predetermined period elapses.

When the accessory power supply or the ignition power supply changes from the OFF state to the ON state, first, in step ST601, the learned model acquiring unit 140 acquires learned model information.

Next, in step ST610, for example, a power supply determining unit (not illustrated in FIG. 2) included in the awakening level estimation device 100 determines whether or not the accessory power supply or the ignition power supply has changed from the ON state to the OFF state.

In step ST610, if the power supply determining unit determines that the accessory power supply or the ignition power supply has changed from the ON state to the OFF state, the awakening level estimation device 100 ends the processing of the flowchart.

In step ST610, if the power supply determining unit determines that the accessory power supply or the ignition power supply has not changed from the ON state to the OFF state, that is, if the power supply determining unit determines that the accessory power supply or the ignition power supply remains in the ON state, the awakening level estimation device 100 executes processing in step ST611 and subsequent steps described below.

In step ST610, if the power supply determining unit determines that the accessory power supply or the ignition power supply has not changed from the ON state to the OFF state, that is, if the power supply determining unit determines that the accessory power supply or the ignition power supply remains in the ON state, first, in step ST611, the sensor signal acquiring unit 101 acquires a sensor signal.

Next, in step ST612, the image acquiring unit 102 acquires image information.

Next, in step ST613, the occupant state acquiring unit 110 acquires n-th occupant state information.

Next, in step ST620, the occupant basic state acquiring unit 120 determines whether or not n-th occupant basic state information has already been acquired.

In step ST620, if the occupant basic state acquiring unit 120 determines that the n-th occupant basic state information has not been acquired yet, in step ST630, the occupant basic state acquiring unit 120 determines whether or not a predetermined period has elapsed from start of new traveling of the vehicle 1.

In step ST630, if the occupant basic state acquiring unit 120 determines that a predetermined period has not elapsed from start of new traveling of the vehicle 1, the awakening level estimation device 100 returns to the processing in step ST610 and executes the processing in step ST610.

In step ST630, if the occupant basic state acquiring unit 120 determines that a predetermined period has elapsed from start of new traveling of the vehicle 1, in step ST631, the occupant basic state acquiring unit 120 acquires n-th occupant state information as n-th occupant basic state information.

After step ST631, the awakening level estimation device 100 returns to the processing in step ST610 and executes the processing in step ST610.

In step ST620, if the occupant basic state acquiring unit 120 determines that the n-th occupant basic state information has already been acquired, the awakening level estimation device 100 executes processing in step ST621 and subsequent steps.

In step ST620, if the occupant basic state acquiring unit 120 determines that the n-th occupant basic state information has already been acquired, first, in step ST621, the difference acquiring unit 130 acquires n-th difference information.

Next, in step ST622, the awakening level estimating unit 150 generates and outputs awakening level information.

After step ST622, in step ST640, the control signal generating unit 190 determines whether or not the awakening level indicated by the awakening level information is equal to or more than the awakening threshold.

In step ST640, if the control signal generating unit 190 determines that the awakening level indicated by the awakening level information is equal to or more than the awakening threshold, the awakening level estimation device 100 returns to the processing in step ST610 and executes the processing in step ST610.

In step ST640, if the control signal generating unit 190 determines that the awakening level indicated by the awakening level information is not equal to or more than the awakening threshold, that is, if the control signal generating unit 190 determines that the awakening level indicated by the awakening level information is less than the awakening threshold, in step ST641, the control signal generating unit 190 generates a control signal.

After step ST641, in step ST642, the control signal output unit 199 outputs the control signal.

After step ST642, the awakening level estimation device 100 returns to the processing in step ST610 and executes the processing in step ST610.

In the flowchart illustrated in FIG. 6, the processing in step ST611 and the processing in step ST612 may be executed in any order.

As described above, the awakening level estimation device 100 according to the first embodiment includes: the occupant state acquiring unit 110 that acquires two or more types of occupant state information different from each other, the occupant state information indicating a current state value which is a state value of an occupant of the vehicle 1; the occupant basic state acquiring unit 120 that acquires occupant basic state information corresponding to each of the two or more types of occupant state information acquired by the occupant state acquiring unit 110 and indicating a basic state value which is the state vale of the occupant when the occupant is in an awakening state; the difference acquiring unit 130 that acquires difference information indicating a difference between the current state value indicated by the occupant state information acquired by the occupant state acquiring unit 110 and the basic state value indicated by the occupant basic state information acquired by the occupant basic state acquiring unit 120 and corresponding to the occupant state information, the difference acquiring unit 130 acquiring two or more types of difference information corresponding to the two or more types of occupant state information acquired by the occupant state acquiring unit 110; and the awakening level estimating unit 150 that estimates an awakening level of the occupant on the basis of the two or more types of difference information acquired by the difference acquiring unit 130, the awakening level estimating unit 150 inputting the two or more types of difference information to a learned model corresponding to a leaning result of machine learning, and generating and outputting awakening level information indicating an awakening level on the basis of an estimation result output from the leaned model.

With such a configuration, the awakening level estimation device 100 can estimate the awakening level of an occupant using two or more types of occupant state information.

In particular, the current state value indicated by the occupant state information indicates a value depending on each occupant. However, the awakening level estimation device 100 estimates the awakening level of the occupant on the basis of the difference information, and therefore can estimate the awakening level of any one of occupants.

In addition, as described above, the awakening level estimation device 100 according to the first embodiment is configured to include the image acquiring unit 102 that acquires image information output from the imaging device 12 that outputs image information indicating an image obtained by imaging the occupant in addition to the above-described configuration, in which the occupant state acquiring unit 110 acquires at least one type of occupant state information out of two or more types of occupant state information different from each other on the basis of the image information acquired by the image acquiring unit 102.

With such a configuration, the awakening level estimation device 100 can estimate the awakening level of an occupant using two or more types of occupant state information.

In addition, as described above, the awakening level estimation device 100 according to the first embodiment is configured to include the sensor signal acquiring unit 101 that acquires a sensor signal output from the biometric sensor 11 that outputs a sensor signal obtained by detecting a state related to a living body of an occupant in addition to the above-described configuration, in which the occupant state acquiring unit 110 acquires at least one type of occupant state information out of two or more types of occupant state information different from each other on the basis of the sensor signal acquired by the sensor signal acquiring unit 101.

With such a configuration, the awakening level estimation device 100 can estimate the awakening level of an occupant using two or more types of occupant state information.

In addition, as described above, in the above-described configuration, the awakening level estimation device 100 according to the first embodiment is configured in such a manner that the current state value indicated by the occupant state information acquired by the occupant state acquiring unit 110 on the basis of the sensor signal is at least any one of a heart rate per unit time, a heart rate variation value in a predetermined period, a respiration rate per unit time, a respiration cycle in a predetermined period, and a body temperature.

With such a configuration, the awakening level estimation device 100 can estimate the awakening level of an occupant using two or more types of occupant state information.

In addition, as described above, in the above-described configuration, the awakening level estimation device 100 according to the first embodiment is configured in such a manner that the occupant basic state acquiring unit 120 acquires, as occupant basic state information, information indicating a statistical value of a current state value indicated by occupant state information acquired by the occupant state acquiring unit 110 in a period from start of new traveling of the vehicle 1 until a predetermined period elapses.

With such a configuration, the awakening level estimation device 100 can estimate the awakening level of an occupant using two or more types of occupant state information.

In particular, with such a configuration, the awakening level estimation device 100 can estimate the awakening level of the occupant using two or more types of occupant state information without creating the occupant basic state information in advance.

In addition, as described above, the awakening level estimation device 100 according to the first embodiment is configured to include the occupant identifying unit 160 that identifies an occupant and acquires personal identification information indicating the identified person in addition to the above-described configuration, in which the occupant basic state acquiring unit 120 acquires occupant basic state information corresponding to the personal identification information on the basis of the personal identification information acquired by the occupant identifying unit 160, and the occupant basic state information acquired by the occupant basic state acquiring unit 120 is information indicating a statistical value of a current state value indicated by the occupant state information of the occupant indicated by the personal identification information, acquired by the occupant state acquiring unit 110 in a period in which the occupant is in an awakening state in a period in which the occupant was in the vehicle 1 in the past.

With such a configuration, the awakening level estimation device 100 can estimate the awakening level of an occupant using two or more types of occupant state information.

In particular, with such a configuration, the awakening level estimation device 100 can estimate the awakening level of the occupant using two or more types of occupant state information without any time delay from start of new traveling of the vehicle 1.

In addition, as described above, in the above-described configuration, the awakening level estimation device 100 according to the first embodiment is configured in such a manner that the occupant basic state information acquired by the occupant basic state acquiring unit 120 is information indicating a statistical value of a current state value indicated by the occupant state information acquired by the occupant state acquiring unit 110 in a period excluding a period of a special travel state which is a predetermined travel state in the travel state of the vehicle 1.

With such a configuration, the awakening level estimation device 100 can estimate the awakening level of an occupant using two or more types of occupant state information.

In particular, with such a configuration, the awakening level estimation device 100 acquires the occupant basic state information on the basis of the occupant state information acquired in a period excluding a period of the special travel state of the vehicle 1, and therefore accuracy of the occupant basic state information can be enhanced.

In addition, as described above, in the above-described configuration, the awakening level estimation device 100 according to the first embodiment is configured in such a manner that the period of the special travel state includes at least any one of a stop period, a lane change period, a right/left turning period, a conversation period, a congestion period, an initial view travel period, a narrow travel period, a predetermined time period travel period, a predetermined weather travel period, and a complicated driving period.

With such a configuration, the awakening level estimation device 100 can estimate the awakening level of an occupant using two or more types of occupant state information.

In particular, with such a configuration, the awakening level estimation device 100 acquires the occupant basic state information on the basis of the occupant state information acquired in a period excluding a period of the special travel state of the vehicle 1, and therefore accuracy of the occupant basic state information can be enhanced.

In addition, as described above, in the above-described configuration, the awakening level estimation device 100 according to the first embodiment is configured in such a manner that the predetermined driving operation includes at least any one of a steering wheel operation, an accelerator operation, a brake operation, and a horn operation.

With such a configuration, the awakening level estimation device 100 can estimate the awakening level of an occupant using two or more types of occupant state information.

In particular, with such a configuration, the awakening level estimation device 100 acquires the occupant basic state information on the basis of the occupant state information acquired in a period excluding a period of the special travel state of the vehicle 1, and therefore accuracy of the occupant basic state information can be enhanced.

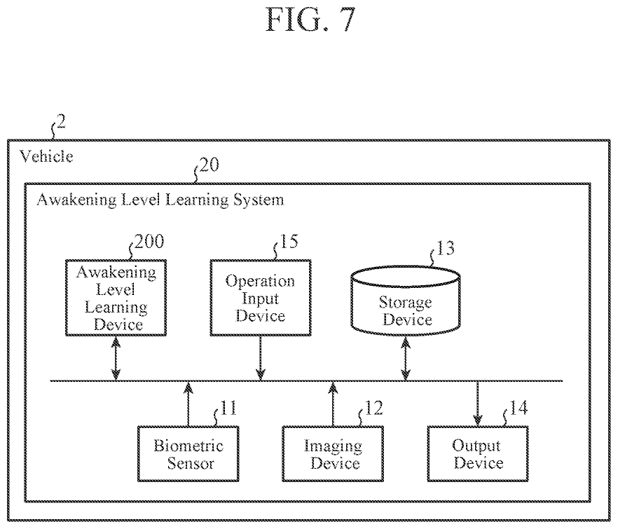

An awakening level learning device 200 according to the first embodiment will be described with reference to FIGS. 7 to 11.

With reference to FIG. 7, a configuration of a main part of an awakening level learning system 20 to which the awakening level learning device 200 according to the first embodiment is applied will be described.

FIG. 7 is a block diagram illustrating an example of the configuration of the main part of the awakening level learning system 20 to which the awakening level learning device 200 according to the first embodiment is applied.

The awakening level learning system 20 is mounted, for example, on a vehicle 2 different from the vehicle 1. The awakening level learning system 20 may be mounted on the vehicle 1.

Hereinafter, the awakening level learning system 20 will be described as being mounted on the vehicle 2 different from the vehicle 1.

The awakening level learning system 20 includes a biometric sensor 11, an imaging device 12, a storage device 13, an operation input device 15, and the awakening level learning device 200.

In FIG. 7, the same reference numerals are given to components similar to those illustrated in FIG. 1, and detailed description thereof is omitted. That is, detailed description of the biometric sensor 11, the imaging device 12, and the storage device 13 is omitted.

The storage device 13 stores information necessary for the awakening level learning device 200 to execute predetermined processing determined in advance. The awakening level learning device 200 can acquire information by reading the information stored in the storage device 13. In addition, the storage device 13 receives information output from the awakening level learning device 200 and stores the information. The awakening level learning device 200 can store information in the storage device 13 by outputting the information to the storage device 13.

The operation input device 15 receives an operation of an occupant and outputs an operation signal based on the operation. For example, at the time of the operation, the occupant performs an operation of inputting whether or not the occupant is in an awakening state, the awakening level of the occupant, or the like to the operation input device 15. For example, the occupant performs an operation input by tapping the operation input device 15 constituted by a touch panel. The occupant may perform the operation input by performing a voice input to the operation input device 15 constituted by a voice recognition device or the like.

Note that another occupant different from the occupant may perform an operation of inputting whether or not the occupant is in an awakening state, the awakening level of the occupant, or the like to the operation input device 15

In addition, the operation input device 15 does not need to be disposed in the vehicle interior of the vehicle 2, and may be disposed, for example, at a remote place away from the vehicle 2.

For example, in a case where the operation input device 15 is disposed at a remote place away from the vehicle 2, a supervisor monitoring, at the remote place, an image in which an occupant is captured may determine whether or not the occupant is in an awakening state, the awakening level of the occupant, or the like, and the supervisor may input a determination result to the operation input device 15.

The awakening level learning device 200 generates a learned model by receiving a sensor signal output from the biometric sensor 11 and image information output from the imaging device 12, and causing a learning model for estimating the awakening level of the occupant to perform machine learning on the basis of the sensor signal and the image information. The awakening level learning device 200 outputs the generated learned model as learned model information and stores the learned model in the storage device 13.

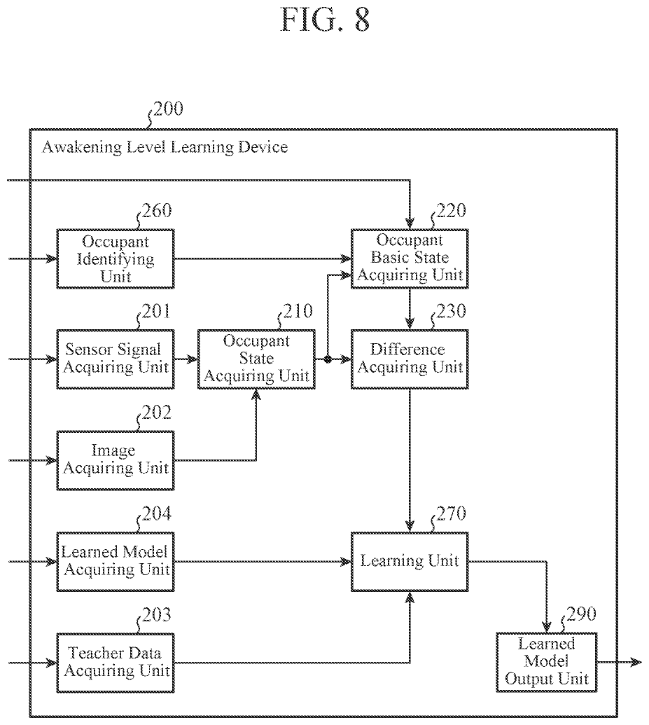

A configuration of a main part of the awakening level learning device 200 according to the first embodiment will be described with reference to FIG. 8.

FIG. 8 is a block diagram illustrating an example of the configuration of the main part of the awakening level learning device 200 according to the first embodiment.

The awakening level learning device 200 includes a sensor signal acquiring unit 201, an image acquiring unit 202, a teacher data acquiring unit 203, a learning model acquiring unit 204, an occupant state acquiring unit 210, an occupant basic state acquiring unit 220, a difference acquiring unit 230, an occupant identifying unit 260, a learning unit 270, and a learned model output unit 290.

The sensor signal acquiring unit 201 acquires a sensor signal output from the biometric sensor 11. Since the sensor signal acquiring unit 201 is similar to the sensor signal acquiring unit 101 included in the awakening level estimation device 100 illustrated in FIG. 2, description thereof is omitted.

The image acquiring unit 202 acquires image information output from the imaging device 12. Since the image acquiring unit 202 is similar to the image acquiring unit 102 included in the awakening level estimation device 100 illustrated in FIG. 2, description thereof is omitted.

The occupant identifying unit 260 identifies an occupant in the vehicle 2, and acquires personal identification information indicating the person. Since the occupant identifying unit 260 is similar to the occupant identifying unit 160 included in the awakening level estimation device 100 illustrated in FIG. 2, description thereof is omitted.

The learning model acquiring unit 204 acquires learning model information indicating a learning model that has not been learned or is being learned.

Specifically, for example, by reading learning model information stored in advance in the storage device 13 from the storage device 13, the learning model acquiring unit 204 acquires the learning model information.

The occupant state acquiring unit 210 acquires two or more types of occupant state information different from each other. Since the occupant state acquiring unit 210 is similar to the occupant state acquiring unit 110 included in the awakening level estimation device 100 illustrated in FIG. 2, description thereof is omitted. That is, the occupant state acquiring unit 210 includes, for example, N feature amount extracting units (not illustrated in FIG. 8), and the occupant state acquiring unit 210 acquires n-th occupant state information.

Note that, in the above description, the awakening level learning device 200 includes the sensor signal acquiring unit 201 and the image acquiring unit 202, but the awakening level learning device 200 may include either one of the sensor signal acquiring unit 201 and the image acquiring unit 202.

For example, in a case where the awakening level learning device 200 includes the sensor signal acquiring unit 201 but does not include the image acquiring unit 202, the occupant state acquiring unit 210 acquires two or more types of occupant state information different from each other on the basis of one or more sensor signals acquired by the sensor signal acquiring unit 201.

For example, in a case where the awakening level learning device 200 includes the image acquiring unit 202 but does not include the sensor signal acquiring unit 201, the occupant state acquiring unit 210 acquires two or more types of occupant state information different from each other on the basis of the image information acquired by the image acquiring unit 202.

In addition, in the above description, the awakening level learning device 200 includes the sensor signal acquiring unit 201 and the image acquiring unit 202, and the occupant state acquiring unit 210 includes N feature amount extracting units. However, the sensor signal acquiring unit 201, the image acquiring unit 202, and the N feature amount extracting units included in the occupant state acquiring unit 210 may be included in an external device (not illustrated in FIG. 7) such as an occupant state acquiring device different from the awakening level learning device 200. In a case where the external device such as the occupant state acquiring device includes the sensor signal acquiring unit 201, the image acquiring unit 202, and the N feature amount extracting units included in the occupant state acquiring unit 210, for example, the occupant state acquiring unit 210 acquires two or more types of occupant state information different from each other by acquiring n-th occupant state information acquired by the external device from the external device.

The teacher data acquiring unit 203 acquires teacher data used when a learning model indicated by learning model information acquired by the learning model acquiring unit 204 is caused to perform machine learning with supervised learning.

Specifically, for example, the teacher data acquiring unit 203 receives an operation signal output from the operation input device 15, and acquires, as the teacher data, information indicating whether or not an occupant corresponding to the operation signal is in an awakening state, the awakening level of the occupant, or the like.

The teacher data acquiring unit 203 is not limited to one that acquires teacher data based on the operation signal output from the operation input device 15.

For example, the teacher data acquiring unit 203 may generate and acquire the teacher data by receiving an electroencephalogram signal of the occupant output from an electroencephalogram measuring device (not illustrated in FIG. 7) that measures electroencephalograms of the occupant, and analyzing whether or not the occupant is in an awakening state, the awakening level of the occupant, or the like on the basis of the electroencephalogram signal.

The occupant basic state acquiring unit 220 acquires occupant basic state information corresponding to each of the two or more types of occupant state information acquired by the occupant state acquiring unit 210. Since the occupant basic state acquiring unit 220 is similar to the occupant basic state acquiring unit 120 included in the awakening level estimation device 100 illustrated in FIG. 2, description thereof is omitted. That is, the occupant basic state acquiring unit 220 acquires n-th occupant basic state information.

Note that the occupant basic state acquiring unit 220 may acquire the occupant basic state information by a method different from the method by which the occupant basic state acquiring unit 120 included in the awakening level estimation device 100 acquires the occupant basic state information. Specifically, for example, the occupant basic state acquiring unit 220 may acquire, as the occupant basic state information, a statistical value such as an average value, a median value, or a mode value of current state values indicated by each of the two or more types of occupant state information acquired by the occupant state acquiring unit 210 in a period in which the teacher data acquired by the teacher data acquiring unit 203 continuously indicates that the occupant is in an awakening state.

The difference acquiring unit 230 acquires difference information indicating a difference between a current state value indicated by the occupant state information acquired by the occupant state acquiring unit 210 and a basic state value indicated by the occupant basic state information acquired by the occupant basic state acquiring unit 220 and corresponding to the occupant state information. Since the difference acquiring unit 230 is similar to the difference acquiring unit 130 included in the awakening level estimation device 100 illustrated in FIG. 2, description thereof is omitted. That is, the difference acquiring unit 230 acquires n-th difference information.

The learning unit 270 inputs, as explanatory variables, two or more types of difference information acquired by the difference acquiring unit 230 to a learning model indicated by the learning model information acquired by the learning model acquiring unit 204, and causes the learning model to perform machine learning with supervised learning based on the teacher data acquired by the teacher data acquiring unit 203. For example, the learning unit 270 generates a learned model by causing the learning model indicated by the learning model information acquired by the learning model acquiring unit 204 to perform machine learning with supervised learning a predetermined number of times or for a predetermined time.

Specifically, the learning unit 270 inputs, as explanatory variables, two or more types of difference information acquired by the difference acquiring unit 230 by causing the learning model to perform machine learning with supervised learning, and generates a learned model that outputs a numerical value indicating the awakening level of the occupant as an estimation result in a predetermined format such as a percentage. The learned model generated by the learning unit 270 causing the learning model to perform machine learning with supervised learning may output, as the estimation result, a reliability of the numerical value in a predetermined format such as a percentage in addition to the numerical value indicating the awakening level of the occupant.

Note that the two or more types of difference information input as explanatory variables to the learning model indicated by the learning model information acquired by the learning model acquiring unit 204 and the two or more types of difference information acquired by the difference acquiring unit 230 and input as explanatory variables to the learned model by the awakening level estimating unit 150 included in the awakening level estimation device 100 are the same type of difference information.

The learned model output unit 290 outputs learned model information indicating the learned model generated by the learning unit 270. Specifically, for example, the learned model output unit 290 outputs the learned model information to the storage device 13, and stores the learned model information in the storage device 13.

With the above configuration, the awakening level learning device 200 can generate a learned model capable of estimating a numerical value indicating the awakening level of an occupant using two or more types of occupant state information.

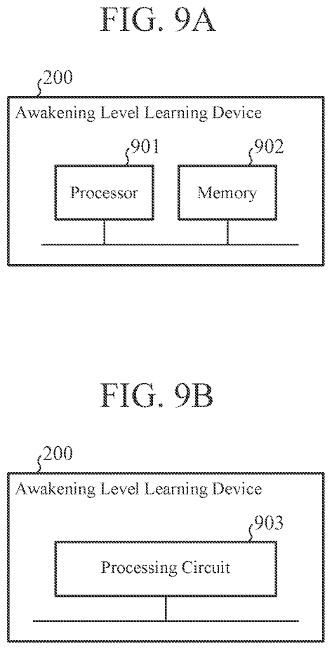

A hardware configuration of a main part of the awakening level learning device 200 according to the first embodiment will be described with reference to FIGS. 9A and 9B.

FIGS. 9A and 9B are diagrams illustrating an example of a hardware configuration of the main part of the awakening level learning device 200 according to the first embodiment.

As illustrated in FIG. 9A, the awakening level learning device 200 is constituted by a computer, and the computer includes a processor 901 and a memory 902. The memory 902 stores a program for causing the computer to function as the sensor signal acquiring unit 201, the image acquiring unit 202, the teacher data acquiring unit 203, the learning model acquiring unit 204, the occupant state acquiring unit 210, the occupant basic state acquiring unit 220, the difference acquiring unit 230, the occupant identifying unit 260, the learning unit 270, and the learned model output unit 290. By the processor 901 reading and executing the program stored in the memory 902, the sensor signal acquiring unit 201, the image acquiring unit 202, the teacher data acquiring unit 203, the learning model acquiring unit 204, the occupant state acquiring unit 210, the occupant basic state acquiring unit 220, the difference acquiring unit 230, the occupant identifying unit 260, the learning unit 270, and the learned model output unit 290 are implemented.

In addition, as illustrated in FIG. 9B, the awakening level learning device 200 may be constituted by a processing circuit 903. In this case, the functions of the sensor signal acquiring unit 201, the image acquiring unit 202, the teacher data acquiring unit 203, the learning model acquiring unit 204, the occupant state acquiring unit 210, the occupant basic state acquiring unit 220, the difference acquiring unit 230, the occupant identifying unit 260, the learning unit 270, and the learned model output unit 290 may be implemented by the processing circuit 903.

Alternatively, the awakening level learning device 200 may be constituted by the processor 901, the memory 902, and the processing circuit 903 (not illustrated). In this case, some of the functions of the sensor signal acquiring unit 201, the image acquiring unit 202, the teacher data acquiring unit 203, the learning model acquiring unit 204, the occupant state acquiring unit 210, the occupant basic state acquiring unit 220, the difference acquiring unit 230, the occupant identifying unit 260, the learning unit 270, and the learned model output unit 290 may be implemented by the processor 901 and the memory 902, and the remaining functions may be implemented by the processing circuit 903.

Note that since the processor 901, the memory 902, and the processing circuit 903 are similar to the processor 401, the memory 402, and the processing circuit 403 illustrated in FIG. 4, respectively, description thereof is omitted.

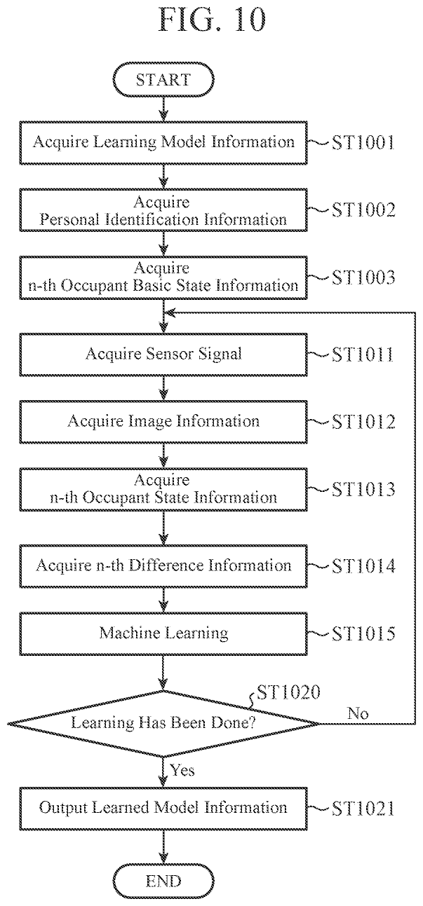

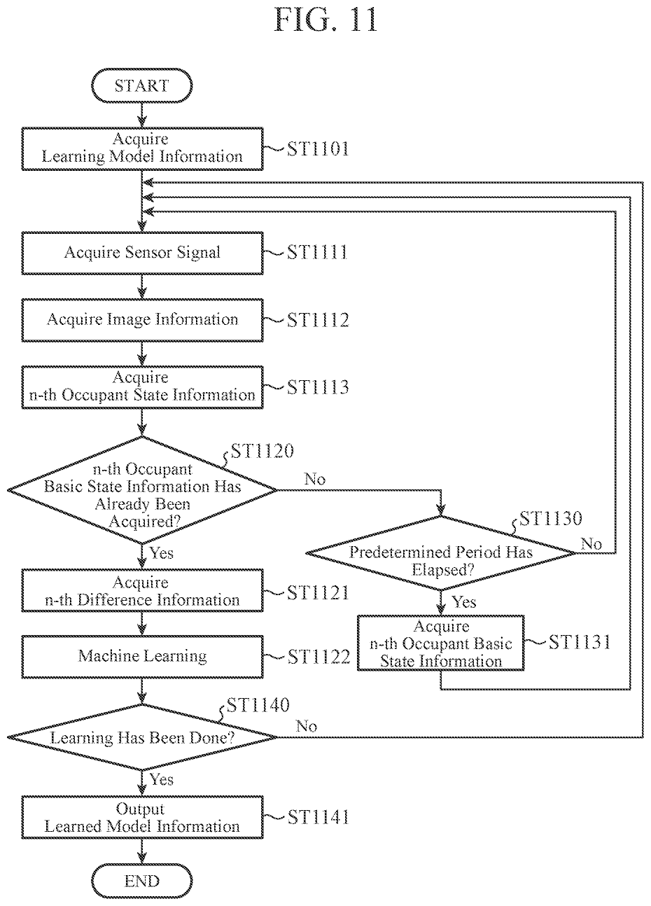

An operation of the awakening level learning device 200 according to the first embodiment will be described with reference to FIGS. 10 and 11.

FIG. 10 is a flowchart for explaining an example of processing of the awakening level learning device 200 according to the first embodiment.

In addition, the flowchart illustrated in FIG. 10 illustrates, as an example, processing of the awakening level learning device 200 in a case where occupant basic state information of an occupant is stored in the storage device 13 in advance.

First, in step ST1001, the learning model acquiring unit 204 acquires learning model information.

Next, in step ST1002, the occupant identifying unit 260 acquires personal identification information.

First, in step ST1003, the occupant basic state acquiring unit 220 acquires n-th occupant basic state information.

Next, in step ST1011, the sensor signal acquiring unit 201 acquires a sensor signal.

Next, in step ST1012, the image acquiring unit 202 acquires image information.

Next, in step ST1013, the occupant state acquiring unit 210 acquires n-th occupant state information.

Next, in step ST1014, the difference acquiring unit 230 acquires n-th difference information.

Next, in step ST1015, the learning unit 270 causes a learning model to perform machine learning with supervised learning.

Next, in step ST1020, the learned model output unit 290 determines whether or not the learning unit 270 has caused the learning model to perform learning a predetermined number of times or for a predetermined time.

In step ST1020, if the learned model output unit 290 determines that the learning unit 270 has not caused the learning model to perform learning a predetermined number of times or for a predetermined time, the awakening level learning device 200 returns to the processing in step ST1011 and executes the processing in step ST1011 and subsequent steps.

In step ST1020, if the learned model output unit 290 determines that the learning unit 270 has caused the learning model to perform learning a predetermined number of times or for a predetermined time, in step ST1021, the learned model output unit 290 outputs learned model information.

After step ST1021, the awakening level learning device 200 ends the processing of the flowchart.

Note that, in the flowchart illustrated in FIG. 10, the processing from step ST1001 to step ST1003 may be executed in any order as long as the processing in step ST1002 is executed before the processing in step ST1003 is executed. In addition, the processing in step ST1011 and the processing in step ST1012 may be executed in any order.

FIG. 11 is a flowchart for explaining another example of the processing of the awakening level learning device 200 according to the first embodiment.

The flowchart illustrated in FIG. 11 indicates, as an example, processing of the awakening level learning device 200 in a case where the occupant basic state acquiring unit 220 acquires, as the k-th occupant basic state information, information indicating a statistical value of a current state value indicated by the k-th occupant state information acquired by the occupant state acquiring unit 210 in a period from start of new traveling of the vehicle 2 until a predetermined period elapses.

First, in step ST1101, the learning model acquiring unit 204 acquires learning model information.

Next, in step ST1111, the sensor signal acquiring unit 201 acquires a sensor signal.

Next, in step ST1112, the image acquiring unit 202 acquires image information.

Next, in step ST1113, the occupant state acquiring unit 210 acquires n-th occupant state information.

Next, in step ST1120, the occupant basic state acquiring unit 220 determines whether or not n-th occupant basic state information has already been acquired.

In step ST1120, if the occupant basic state acquiring unit 220 determines that the n-th occupant basic state information has not been acquired yet, in step ST1130, the occupant basic state acquiring unit 220 determines whether or not a predetermined period has elapsed from start of new traveling of the vehicle 2.

In step ST1130, if the occupant basic state acquiring unit 220 determines that a predetermined period has not elapsed from start of new traveling of the vehicle 2, the awakening level learning device 200 returns to the processing in step ST1111 and executes the processing in step ST1111 and subsequent steps.

In step ST1130, if the occupant basic state acquiring unit 220 determines that a predetermined period has elapsed from start of new traveling of the vehicle 2, the occupant basic state acquiring unit 220 acquires n-th occupant state information as n-th occupant basic state information in step ST1131.

After step ST1131, the awakening level learning device 200 returns to the processing in step ST1111 and executes the processing in step ST1111 and subsequent steps.

In step ST1120, if the occupant basic state acquiring unit 220 determines that n-th occupant basic state information has already been acquired, the awakening level learning device 200 executes processing in step ST1121 and subsequent steps.

In step ST1120, if the occupant basic state acquiring unit 220 determines that n-th occupant basic state information has already been acquired, first, in step ST1121, the difference acquiring unit 230 acquires n-th difference information.

Next, in step ST1122, the learning unit 270 causes the learning model to perform machine learning with supervised learning.

After step ST1122, in step ST1140, the learned model output unit 290 determines whether or not the learning unit 270 has caused the learning model to perform learning a predetermined number of times or for a predetermined time.

In step ST1140, if the learned model output unit 290 determines that the learning unit 270 has not caused the learning model to perform learning a predetermined number of times or for a predetermined time, the awakening level learning device 200 returns to the processing in step ST1111 and executes the processing in step ST1111 and subsequent steps.

In step ST1140, if the learned model output unit 290 determines that the learning unit 270 has caused the learning model to perform learning a predetermined number of times or for a predetermined time, in step ST1141, the learned model output unit 290 outputs learned model information.

After step ST1141, the awakening level learning device 200 ends the processing of the flowchart.

Note that, in the flowchart illustrated in FIG. 11, the processing in step ST1111 and the processing in step ST1112 may be executed in any order.

As described above, the awakening level learning device 200 according to the first embodiment includes: the learning model acquiring unit 204 that acquires learning model information indicating a learning model that has not been learned or is being learned; the teacher data acquiring unit 203 that acquires teacher data used when the learning model indicated by the learning model information acquired by the learning model acquiring unit 204 is caused to perform machine learning with supervised learning; the occupant state acquiring unit 210 that acquires two or more types of occupant state information different from each other, the occupant state information indicating a current state value which is a state value of an occupant of the vehicle 2; the occupant basic state acquiring unit 220 that acquires occupant basic state information corresponding to each of the two or more types of occupant state information acquired by the occupant state acquiring unit 210 and indicating a basic state value which is the state vale of the occupant when the occupant is in an awakening state; the difference acquiring unit 230 that acquires difference information indicating a difference between the current state value indicated by the occupant state information acquired by the occupant state acquiring unit 210 and the basic state value indicated by the occupant basic state information acquired by the occupant basic state acquiring unit 220 and corresponding to the occupant state information, the difference acquiring unit 230 acquiring two or more types of difference information corresponding to the two or more types of occupant state information acquired by the occupant state acquiring unit 210; the learning unit 270 that generates a learned model that outputs information indicating the awakening level of the occupant as an estimation result by inputting, as explanatory variables, the two or more types of difference information acquired by the difference acquiring unit 230 to the learning model indicated by the learning model information acquired by the learning model acquiring unit 204, and causing the learning model to perform machine learning with supervised learning based on the teacher data acquired by the teacher data acquiring unit 203; and the learned model output unit 290 that outputs the learned model generated by the learning unit 270 as learned model information.

With such a configuration, the awakening level learning device 200 can generate a learned model capable of estimating the awakening level of an occupant using two or more types of occupant state information.

In particular, the awakening level learning device 200 causes a learning model that estimates the awakening level of an occupant to perform learning using difference information as an explanatory variable. Therefore, the learned model generated by the awakening level learning device 200 can estimate the awakening level of an occupant without depending on the occupant even if an occupant in the vehicle 2 when the learning model is caused to perform learning is different from an occupant in the vehicle 1 when the awakening level estimation device 100 estimates the awakening level of an occupant.

In addition, as described above, the awakening level learning device 200 according to the first embodiment is configured to include the image acquiring unit 202 that acquires image information output from the imaging device 12 that outputs the image information indicating an image obtained by imaging the occupant in addition to the above-described configuration, in which the occupant state acquiring unit 210 acquires at least one type of occupant state information out of two or more types of occupant state information different from each other on the basis of the image information acquired by the image acquiring unit 202.

With such a configuration, the awakening level learning device 200 can generate a learned model capable of estimating the awakening level of an occupant using two or more types of occupant state information.

In addition, as described above, the awakening level learning device 200 according to the first embodiment is configured to include the sensor signal acquiring unit 201 that acquires a sensor signal output from the biometric sensor 11 that outputs a sensor signal obtained by detecting a state related to a living body of an occupant in addition to the above-described configuration, in which the occupant state acquiring unit 210 acquires at least one type of occupant state information out of two or more types of occupant state information different from each other on the basis of the sensor signal acquired by the sensor signal acquiring unit 201.

With such a configuration, the awakening level learning device 200 can generate a learned model capable of estimating the awakening level of an occupant using two or more types of occupant state information.

In addition, as described above, in the above-described configuration, the awakening level learning device 200 according to the first embodiment is configured in such a manner that the current state value indicated by the occupant state information acquired by the occupant state acquiring unit 210 on the basis of the sensor signal is at least any one of a heart rate per unit time, a heart rate variation value in a predetermined period, a respiration rate per unit time, a respiration cycle in a predetermined period, and a body temperature.

With such a configuration, the awakening level learning device 200 can generate a learned model capable of estimating the awakening level of an occupant using two or more types of occupant state information.

In addition, as described above, in the above-described configuration, the awakening level learning device 200 according to the first embodiment is configured in such a manner that the occupant basic state acquiring unit 220 acquires, as occupant basic state information, information indicating a statistical value of a current state value indicated by occupant state information acquired by the occupant state acquiring unit 210 in a period from start of new traveling of the vehicle 2 until a predetermined period elapses.

With such a configuration, the awakening level learning device 200 can generate a learned model capable of estimating the awakening level of an occupant using two or more types of occupant state information.

In particular, with such a configuration, the awakening level learning device 200 can generate a learned model capable of estimating the awakening level of an occupant using two or more types of occupant state information without creating the occupant basic state information in advance when the learning model is caused to perform learning.

In addition, as described above, the awakening level learning device 200 according to the first embodiment is configured to include the occupant identifying unit 260 that identifies an occupant and acquires personal identification information indicating the identified person in addition to the above-described configuration, in which the occupant basic state acquiring unit 220 acquires occupant basic state information corresponding to the personal identification information on the basis of the personal identification information acquired by the occupant identifying unit 260, and the occupant basic state information acquired by the occupant basic state acquiring unit 220 is information indicating a statistical value of a current state value indicated by the occupant state information of the occupant indicated by the personal identification information, acquired by the occupant state acquiring unit 210 in a period in which the occupant is in an awakening state in a period in which the occupant was in the vehicle 2 in the past.

With such a configuration, the awakening level learning device 200 can generate a learned model capable of estimating the awakening level of an occupant using two or more types of occupant state information.

In particular, with such a configuration, the awakening level learning device 200 can start causing the learning model to perform learning without any time delay from start of new traveling of the vehicle 2.

In addition, as described above, in the above-described configuration, the awakening level learning device 200 according to the first embodiment is configured in such a manner that the occupant basic state information acquired by the occupant basic state acquiring unit 220 is information indicating a statistical value of a current state value indicated by the occupant state information acquired by the occupant state acquiring unit 210 in a period excluding a period of a special travel state which is a predetermined travel state in the travel state of the vehicle 2.

With such a configuration, the awakening level learning device 200 can generate a learned model capable of estimating the awakening level of an occupant using two or more types of occupant state information.

In particular, with such a configuration, the awakening level learning device 200 acquires the occupant basic state information on the basis of the occupant state information acquired in a period excluding a period of the special travel state of the vehicle 2, and therefore can start causing the learning model to perform learning using occupant basic state information with high accuracy.

In addition, as described above, in the above-described configuration, the awakening level learning device 200 according to the first embodiment is configured in such a manner that the period of the special travel state includes at least any one of a stop period, a lane change period, a right/left turning period, a conversation period, a congestion period, an initial view travel period, a narrow travel period, a predetermined time period travel period, a predetermined weather travel period, and a complicated driving period.

With such a configuration, the awakening level learning device 200 can generate a learned model capable of estimating the awakening level of an occupant using two or more types of occupant state information.

In particular, with such a configuration, the awakening level learning device 200 acquires the occupant basic state information on the basis of the occupant state information acquired in a period excluding a period of the special travel state of the vehicle 2, and therefore can start causing the learning model to perform learning using occupant basic state information with high accuracy.

In addition, as described above, in the above-described configuration, the awakening level learning device 200 according to the first embodiment is configured in such a manner that the predetermined driving operation includes at least any one of a steering wheel operation, an accelerator operation, a brake operation, and a horn operation.

With such a configuration, the awakening level learning device 200 can generate a learned model capable of estimating the awakening level of an occupant using two or more types of occupant state information.

In particular, with such a configuration, the awakening level learning device 200 acquires the occupant basic state information on the basis of the occupant state information acquired in a period excluding a period of the special travel state of the vehicle 2, and therefore can start causing the learning model to perform learning using occupant basic state information with high accuracy.

### Second Embodiment

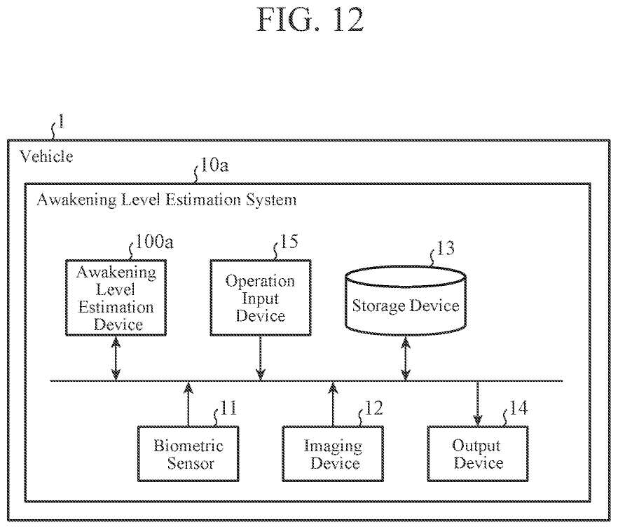

An awakening level estimation device 100a according to a second embodiment will be described with reference to FIGS. 12 to 14.

With reference to FIG. 12, a configuration of a main part of an awakening level estimation system 10a to which the awakening level estimation device 100a according to the second embodiment is applied will be described.

FIG. 12 is a block diagram illustrating an example of the configuration of the main part of the awakening level estimation system 10a to which the awakening level estimation device 100a according to the second embodiment is applied.

The awakening level estimation system 10a is mounted on a vehicle 1.

The awakening level estimation system 10a includes a biometric sensor 11, an imaging device 12, a storage device 13, an output device 14, and the awakening level estimation device 100a.

That is, the awakening level estimation system 10a is obtained by changing the awakening level estimation device 100 according to the first embodiment to the awakening level estimation device 100a.

In FIG. 12, the same reference numerals are given to components similar to those illustrated in FIG. 1, and detailed description thereof is omitted. That is, detailed description of the biometric sensor 11, the imaging device 12, the storage device 13, and the output device 14 is omitted.

The awakening level estimation device 100a includes the function of the awakening level estimation device 100 according to the first embodiment, and additionally includes an additional learning function of causing a learned model used when the awakening level estimation device 100 estimates the awakening level of an occupant to perform additional learning.

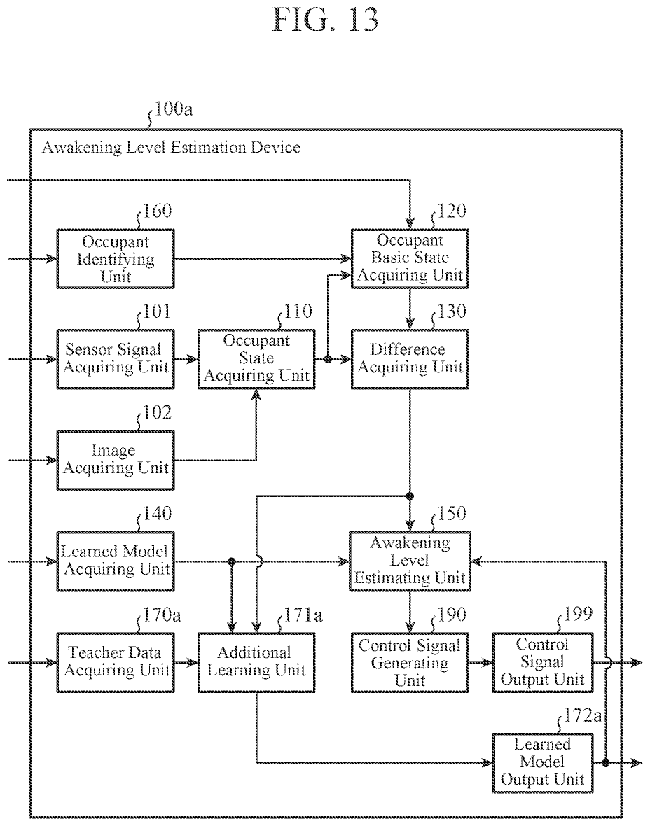

A configuration of a main part of the awakening level estimation device 100a according to the second embodiment will be described with reference to FIG. 13.

FIG. 13 is a block diagram illustrating an example of the configuration of the main part of the awakening level estimation device 100a according to the second embodiment.

The awakening level estimation device 100a includes a sensor signal acquiring unit 101, an image acquiring unit 102, an occupant state acquiring unit 110, an occupant basic state acquiring unit 120, a difference acquiring unit 130, a learned model acquiring unit 140, an awakening level estimating unit 150, an occupant identifying unit 160, a teacher data acquiring unit 170a, an additional learning unit 171a, a learned model output unit 172a, a control signal generating unit 190, and a control signal output unit 199.

That is, the awakening level estimation device 100a is obtained by adding the teacher data acquiring unit 170a, the additional learning unit 171a, and the learned model output unit 172a as compared with the awakening level estimation device 100 according to the first embodiment.

In FIG. 13, the same reference numerals are given to components similar to those illustrated in FIG. 2, and detailed description thereof is omitted. That is, detailed description of the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the control signal generating unit 190, and the control signal output unit 199 is omitted.

Note that the awakening level estimation device 100a does not necessarily need to include the occupant identifying unit 160.

The teacher data acquiring unit 170a acquires teacher data used when a learning model indicated by learned model information acquired by the learned model acquiring unit 140 is caused to perform additional machine learning with supervised learning.

Specifically, for example, the teacher data acquiring unit 170a receives an operation signal output from the operation input device 15, and acquires, as the teacher data, information indicating whether or not an occupant corresponding to the operation signal is in an awakening state, the awakening level of the occupant, or the like.

The additional learning unit 171a inputs, as explanatory variables, two or more types of difference information acquired by the difference acquiring unit 130 to a learned model indicated by learned model information acquired by the learned model acquiring unit 140, and causes the learned model to perform additional machine learning with supervised learning based on teacher data acquired by the teacher data acquiring unit 170a. For example, the additional learning unit 171a updates the learned model by causing the learned model to perform additional machine learning with supervised learning, and uses the updated learned model as a new learned model.

The learned model output unit 172a outputs learned model information indicating the updated learned model updated by the additional learning unit 171a. Specifically, for example, the learned model output unit 172a outputs the learned model information to the storage device 13, and stores the learned model information in the storage device 13.

In addition, the learned model output unit 172a may output the learned model information indicating the updated learned model updated by the additional learning unit 171a to the awakening level estimating unit 150. For example, the awakening level estimating unit 150 receives the updated learned model information output from the learned model output unit 172a, and generates and outputs awakening level information indicating the awakening level on the basis of an estimation result output from the learned model.

With the above configuration, by causing the learned model generated by the awakening level learning device 200 to perform additional learning, the awakening level estimation device 100a can update the learned model and can generate a learned model capable of estimating the awakening level of an occupant with higher accuracy than the learned model.

As a result, the awakening level estimation device 100a can estimate the awakening level of an occupant using two or more types of occupant state information with higher accuracy.

Note that the functions of the sensor signal acquiring unit 101, the image acquiring unit 102, the occupant state acquiring unit 110, the occupant basic state acquiring unit 120, the difference acquiring unit 130, the learned model acquiring unit 140, the awakening level estimating unit 150, the occupant identifying unit 160, the teacher data acquiring unit 170a, the additional learning unit 171a, the learned model output unit 172a, the control signal generating unit 190, and the control signal output unit 199 in the awakening level estimation device 100a according to the second embodiment may be implemented by the processor 401 and the memory 402 or may be implemented by the processing circuit 403 in the hardware configuration illustrated as an example in FIGS. 4A and 4B in the first embodiment.

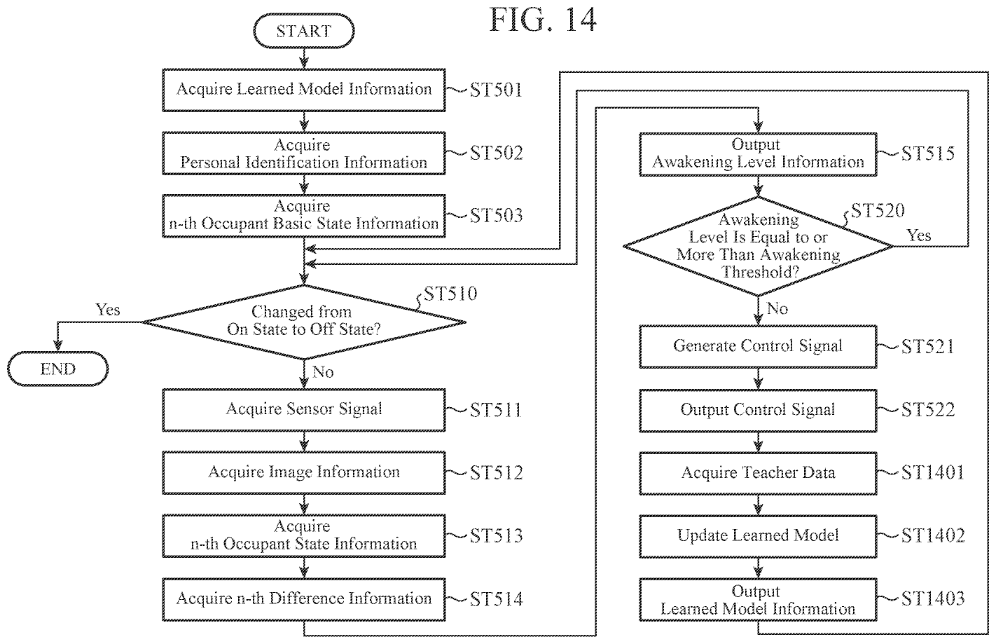

An operation of the awakening level estimation device 100a according to the second embodiment will be described with reference to FIG. 14.

FIG. 14 is a flowchart for explaining an example of processing of the awakening level estimation device 100a according to the second embodiment.

Note that, for example, the awakening level estimation device 100a starts processing of the flowchart when an accessory power supply or an ignition power supply changes from an OFF state to an ON state, and ends the processing of the flowchart when the accessory power supply or the ignition power supply changes from the ON state to the OFF state.

In addition, the flowchart illustrated in FIG. 14 illustrates, as an example, processing of the awakening level estimation device 100a in a case where occupant basic state information for each occupant is stored in the storage device 13 in advance.

Note that, in FIG. 14, the same reference numeral is given to processing similar to that illustrated in FIG. 5, and detailed description thereof is omitted. That is, detailed description of processing in steps ST501 to ST503, steps ST510 to ST515, and steps ST520 to ST522 is omitted.

The flowchart illustrated in FIG. 14 is obtained by adding processing in step ST1401 and subsequent steps after step ST522 in the flowchart illustrated in FIG. 5.

When the accessory power supply or the ignition power supply changes from the OFF state to the ON state, first, in step ST501, the learned model acquiring unit 140 acquires learned model information.

Next, in step ST502, the occupant identifying unit 160 acquires personal identification information.

Next, in step ST503, the occupant basic state acquiring unit 120 acquires n-th occupant basic state information.

Next, in step ST510, for example, a power supply determining unit (not illustrated in FIG. 13) included in the awakening level estimation device 100a determines whether or not the accessory power supply or the ignition power supply has changed from the ON state to the OFF state.

In step ST510, if the power supply determining unit determines that the accessory power supply or the ignition power supply has changed from the ON state to the OFF state, the awakening level estimation device 100a ends the processing of the flowchart.

In step ST510, if the power supply determining unit determines that the accessory power supply or the ignition power supply has not changed from the ON state to the OFF state, that is, if the power supply determining unit determines that the accessory power supply or the ignition power supply remains in the ON state, the awakening level estimation device 100a executes processing in step ST511 and subsequent steps described below.

In step ST510, if the power supply determining unit determines that the accessory power supply or the ignition power supply has not changed from the ON state to the OFF state, that is, if the power supply determining unit determines that the accessory power supply or the ignition power supply remains in the ON state, first, in step ST511, the sensor signal acquiring unit 101 acquires a sensor signal.

Next, in step ST512, the image acquiring unit 102 acquires image information.

Next, in step ST513, the occupant state acquiring unit 110 acquires n-th occupant state information.

Next, in step ST514, the difference acquiring unit 130 acquires n-th difference information.

Next, in step ST515, the awakening level estimating unit 150 generates and outputs awakening level information.

Next, in step ST520, the control signal generating unit 190 determines whether or not the awakening level indicated by the awakening level information is equal to or more than the awakening threshold.

In step ST520, if the control signal generating unit 190 determines that the awakening level indicated by the awakening level information is equal to or more than the awakening threshold, the awakening level estimation device 100a returns to the processing in step ST510 and executes the processing in step ST510.

In step ST520, if the control signal generating unit 190 determines that the awakening level indicated by the awakening level information is not equal to or more than the awakening threshold, that is, if the control signal generating unit 190 determines that the awakening level indicated by the awakening level information is less than the awakening threshold, in step ST521, the control signal generating unit 190 generates a control signal.

After step ST521, in step ST522, the control signal output unit 199 outputs a control signal.

After step ST522, in step ST1401, the teacher data acquiring unit 170a acquires teacher data.

After step ST1401, in step ST1402, the additional learning unit 171a causes the learned model to perform additional machine learning with supervised learning, and updates the learned model.

After step ST1402, in step ST1403, the learned model output unit 172a outputs learned model information indicating the updated learned model.

After step ST1403, the awakening level estimation device 100a returns to the processing in step ST510 and executes the processing in step ST510.

In the flowchart illustrated in FIG. 14, processing in step ST1401 to step ST1403 may be executed at an any timing as long as the order of the processing in step ST1401 to step ST1403 is the order of the flowchart and the processing in step ST1402 is executed after the processing in step ST514.

In addition, the flowchart illustrated in FIG. 14 is obtained by adding the processing in step ST1401 to step ST1403 illustrated in FIG. 14 to the flowchart illustrated in FIG. 5, but it goes without saying that the processing in step ST1401 to step ST1403 illustrated in FIG. 14 can be appropriately added to the flowchart illustrated in FIG. 6.

With the above configuration, by causing the learned model generated by the awakening level learning device 200 to perform additional learning, the awakening level estimation device 100a can update the learned model and can generate a learned model capable of estimating the awakening level of an occupant with higher accuracy than the learned model.

As a result, the awakening level estimation device 100a can estimate the awakening level of an occupant using two or more types of occupant state information with higher accuracy.

Note that any component in the embodiments can be modified, or any component in the embodiments can be omitted within the scope of the present disclosure.

### INDUSTRIAL APPLICABILITY

The awakening level estimation device according to the present disclosure can be applied to an awakening level estimation system.

### REFERENCE SIGNS LIST

1, 2: vehicle, 10, 10a: awakening level estimation system, 11: biometric sensor, 12: imaging device, 13: storage device, 14: output device, 15: operation input device, 100, 100a: awakening level estimation device, 101: sensor signal acquiring unit, 102: image acquiring unit, 110: occupant state acquiring unit, 111, 1111, 1112, 111N: feature amount extracting unit, 120: occupant basic state acquiring unit, 130: difference acquiring unit, 140: learned model acquiring unit, 150: awakening level estimating unit, 160: occupant identifying unit, 170a: teacher data acquiring unit, 171a: additional learning unit, 172a: learned model output unit, 190: control signal generating unit, 199: control signal output unit, 20: awakening level learning system, 200: awakening level learning device, 201: sensor signal acquiring unit, 202: image acquiring unit, 203: teacher data acquiring unit, 204: learning model acquiring unit, 210: occupant state acquiring unit, 220: occupant basic state acquiring unit, 230: difference acquiring unit, 260: occupant identifying unit, 270: learning unit, 290: learned model output unit, 401, 901: processor, 402, 902: memory, 403, 903: processing circuit

1-20. (canceled)

21. An awakening level estimation device comprising:
processing circuitry configured to
acquire two or more types of occupant state information different from each other, the occupant state information indicating a current state value which is a state value of an occupant of a vehicle;
to acquire occupant basic state information corresponding to each of the two or more types of occupant state information having been acquired and indicating a basic state value which is the state vale of the occupant when the occupant is in an awakening state;
acquire difference information indicating a difference between the current state value indicated by the acquired occupant state information and the basic state value indicated by the acquired occupant basic state information and corresponding to the occupant state information, and acquire two or more types of the difference information corresponding to the two or more types of occupant state information having been acquired;
estimate an awakening level of the occupant on a basis of the two or more types of difference information having been acquired, and input the two or more types of difference information to a learned model corresponding to a leaning result by machine learning, and generating and outputting awakening level information indicating the awakening level on a basis of an estimation result output from the leaned model; and
acquire, as the occupant basic state information, information indicating a statistical value of the current state value indicated by the acquired occupant state information in a period from start of new traveling of the vehicle until a predetermined period elapses.

22. The awakening level estimation device according to claim 21, wherein the acquired occupant basic state information is information indicating the statistical value of the current state value indicated by the acquired occupant state information in a period excluding a period of a special travel state which is a predetermined travel state in a travel state of the vehicle.

23. The awakening level estimation device according to claim 22, wherein the period of the special travel state includes at least any one of a stop period, a lane change period, a right/left turning period, a conversation period, a congestion period, an initial view travel period, a narrow travel period, a predetermined time period travel period, a predetermined weather travel period, and a complicated driving period.

24. The awakening level estimation device according to claim 23, wherein a driving operation performed in the complicated driving period includes at least any one of a steering wheel operation, an accelerator operation, a brake operation, and a horn operation.

25. An awakening level estimation device, comprising:
processing circuitry configured to
acquire two or more types of occupant state information different from each other, the occupant state information indicating a current state value which is a state value of an occupant of a vehicle;
acquire occupant basic state information corresponding to each of the two or more types of occupant state information having been acquired and indicating a basic state value which is the state vale of the occupant when the occupant is in an awakening state;
acquire difference information indicating a difference between the current state value indicated by the acquired occupant state information and the basic state value indicated by the acquired occupant basic state information and corresponding to the occupant state information, and acquire two or more types of the difference information corresponding to the two or more types of occupant state information having been acquired; and
estimate an awakening level of the occupant on a basis of the two or more types of difference information having been acquired, and input the two or more types of difference information to a learned model corresponding to a leaning result by machine learning, and generating and outputting awakening level information indicating the awakening level on a basis of an estimation result output from the leaned model; and
identify the occupant and to acquire personal identification information indicating the identified person,
wherein the processing circuitry acquires the occupant basic state information corresponding to the personal identification information on a basis of the acquired personal identification information, and
the acquired occupant basic state information is information indicating a statistical value of the current state value indicated by the acquired occupant state information of the occupant indicated by the personal identification information, in a period in which the occupant is in the awakening state out of periods in which the occupant was in the vehicle in the past.

26. The awakening level estimation device according to claim 25, wherein the acquired occupant basic state information is information indicating the statistical value of the current state value indicated by the acquired occupant state information in a period excluding a period of a special travel state which is a predetermined travel state in a travel state of the vehicle.

27. The awakening level estimation device according to claim 26, wherein the period of the special travel state includes at least any one of a stop period, a lane change period, a right/left turning period, a conversation period, a congestion period, an initial view travel period, a narrow travel period, a predetermined time period travel period, a predetermined weather travel period, and a complicated driving period.

28. The awakening level estimation device according to claim 27, wherein a driving operation performed in the complicated driving period includes at least any one of a steering wheel operation, an accelerator operation, a brake operation, and a horn operation.

29. An awakening level estimation method comprising:
acquiring two or more types of occupant state information different from each other, the occupant state information indicating a current state value which is a state value of an occupant of a vehicle;
acquiring occupant basic state information corresponding to each of the two or more types of occupant state information acquired by the occupant state acquiring step and indicating a basic state value which is the state vale of the occupant when the occupant is in an awakening state;
acquiring difference information indicating a difference between the current state value indicated by the occupant state information acquired by the occupant state acquiring step and the basic state value indicated by the occupant basic state information acquired by the occupant basic state acquiring step and corresponding to the occupant state information, in which two or more types of the difference information corresponding to the two or more types of occupant state information acquired by the occupant state acquiring step is acquired;
estimating an awakening level of the occupant on a basis of the two or more types of difference information acquired by the difference acquiring step, in which the two or more types of difference information are input to a learned model corresponding to a leaning result by machine learning, and awakening level information indicating the awakening level is generated and output on a basis of an estimation result output from the leaned model; and
acquiring, as the occupant basic state information, information indicating a statistical value of the current state value indicated by the occupant state information acquired by the occupant state acquiring step in a period from start of new traveling of the vehicle until a predetermined period elapses.

30. An awakening level estimation method comprising:
acquiring two or more types of occupant state information different from each other, the occupant state information indicating a current state value which is a state value of an occupant of a vehicle;
acquiring occupant basic state information corresponding to each of the two or more types of occupant state information acquired by the occupant state acquiring step and indicating a basic state value which is the state vale of the occupant when the occupant is in an awakening state;
acquiring difference information indicating a difference between the current state value indicated by the occupant state information acquired by the occupant state acquiring step and the basic state value indicated by the occupant basic state information acquired by the occupant basic state acquiring step and corresponding to the occupant state information, in which two or more types of the difference information corresponding to the two or more types of occupant state information acquired by the occupant state acquiring step is acquired;
estimating an awakening level of the occupant on a basis of the two or more types of difference information acquired by the difference acquiring step, in which the two or more types of difference information are input to a learned model corresponding to a leaning result by machine learning, and awakening level information indicating the awakening level is generated and output on a basis of an estimation result output from the leaned model;
identifying the occupant and acquiring personal identification information indicating the identified person; and
acquiring the occupant basic state information corresponding to the personal identification information on a basis of the personal identification information acquired by the occupant identifying step,
wherein the occupant basic state information acquired by the occupant basic state acquiring step is information indicating a statistical value of the current state value indicated by the occupant state information of the occupant indicated by the personal identification information, acquired by the occupant state acquiring step in a period in which the occupant is in the awakening state out of periods in which the occupant was in the vehicle in the past.

31. An awakening level learning device comprising:
processing circuitry configured to
acquire learning model information indicating a learning model that has not been learned or is being learned;
acquire teacher data used when the learning model indicated by the acquired learning model information is caused to perform machine learning with supervised learning;
acquire two or more types of occupant state information different from each other, the occupant state information indicating a current state value which is a state value of an occupant of a vehicle;
acquire occupant basic state information corresponding to each of the two or more types of occupant state information having been acquired and indicating a basic state value which is the state vale of the occupant when the occupant is in an awakening state;
acquire difference information indicating a difference between the current state value indicated by the acquired occupant state information and the basic state value indicated by the acquired occupant basic state information and corresponding to the occupant state information, and acquire two or more types of the difference information corresponding to the two or more types of occupant state information having been acquired;
generate a learned model for outputting information indicating the awakening level of the occupant as an estimation result by inputting, as explanatory variables, the two or more types of difference information having been acquired to the learning model indicated by the acquired learning model information, and causing the learning model to perform machine learning with supervised learning based on the acquired teacher data;
output the generated learned model as learned model information; and
acquire, as the occupant basic state information, information indicating a statistical value of the current state value indicated by the acquired occupant state information in a period from start of new traveling of the vehicle until a predetermined period elapses.

32. The awakening level learning device according to claim 31, wherein the acquired occupant basic state information is information indicating the statistical value of the current state value indicated by the acquired occupant state information in a period excluding a period of a special travel state which is a predetermined travel state in a travel state of the vehicle.

33. The awakening level learning device according to claim 32, wherein the period of the special travel state includes at least any one of a stop period, a lane change period, a right/left turning period, a conversation period, a congestion period, an initial view travel period, a narrow travel period, a predetermined time period travel period, a predetermined weather travel period, and a complicated driving period.

34. The awakening level learning device according to claim 33, wherein a driving operation performed in the complicated driving period includes at least any one of a steering wheel operation, an accelerator operation, a brake operation, and a horn operation.

35. An awakening level learning device comprising:
processing circuitry configured to
acquire learning model information indicating a learning model that has not been learned or is being learned;
acquire teacher data used when the learning model indicated by the acquired learning model information is caused to perform machine learning with supervised learning;
acquire two or more types of occupant state information different from each other, the occupant state information indicating a current state value which is a state value of an occupant of a vehicle;
acquire occupant basic state information corresponding to each of the two or more types of occupant state information having been acquired and indicating a basic state value which is the state vale of the occupant when the occupant is in an awakening state;
acquire difference information indicating a difference between the current state value indicated by the acquired occupant state information and the basic state value indicated by the acquired occupant basic state information and corresponding to the occupant state information, and acquire two or more types of the difference information corresponding to the two or more types of occupant state information having been acquired;
generate a learned model for outputting information indicating the awakening level of the occupant as an estimation result by inputting, as explanatory variables, the two or more types of difference information having been acquired to the learning model indicated by the acquired learning model information, and causing the learning model to perform machine learning with supervised learning based on the acquired teacher data;
output the generated learned model as learned model information; and
identify the occupant and to acquire personal identification information indicating the identified person,
wherein the processing circuitry acquires the occupant basic state information corresponding to the personal identification information on a basis of the acquired personal identification information, and
the acquired occupant basic state information is information indicating a statistical value of the current state value indicated by the acquired occupant state information of the occupant indicated by the personal identification information, in a period in which the occupant is in the awakening state out of periods in which the occupant was in the vehicle in the past.

36. The awakening level learning device according to claim 35, wherein the acquired occupant basic state information is information indicating the statistical value of the current state value indicated by the acquired occupant state information in a period excluding a period of a special travel state which is a predetermined travel state in a travel state of the vehicle.

37. The awakening level learning device according to claim 36, wherein the period of the special travel state includes at least any one of a stop period, a lane change period, a right/left turning period, a conversation period, a congestion period, an initial view travel period, a narrow travel period, a predetermined time period travel period, a predetermined weather travel period, and a complicated driving period.

38. The awakening level learning device according to claim 37, wherein a driving operation performed in the complicated driving period includes at least any one of a steering wheel operation, an accelerator operation, a brake operation, and a horn operation.

39. An awakening level learning method comprising:
acquiring learning model information indicating a learning model that has not been learned or is being learned;
acquiring teacher data used when the learning model indicated by the learning model information acquired by the learning model acquiring step is caused to perform machine learning with supervised learning;
acquiring two or more types of occupant state information different from each other, the occupant state information indicating a current state value which is a state value of an occupant of a vehicle;
acquiring occupant basic state information corresponding to each of the two or more types of occupant state information acquired by the occupant state acquiring step and indicating a basic state value which is the state vale of the occupant when the occupant is in an awakening state;
acquiring difference information indicating a difference between the current state value indicated by the occupant state information acquired by the occupant state acquiring step and the basic state value indicated by the occupant basic state information acquired by the occupant basic state acquiring step and corresponding to the occupant state information, in which two or more types of the difference information corresponding to the two or more types of occupant state information acquired by the occupant state acquiring step are acquired;
generating a learned model for outputting information indicating the awakening level of the occupant as an estimation result by inputting, as explanatory variables, the two or more types of difference information acquired by the difference acquiring step to the learning model indicated by the learning model information acquired by the learning model acquiring step, and causing the learning model to perform machine learning with supervised learning based on the teacher data acquired by the teacher data acquiring step;
outputting the learned model generated by the learning step as learned model information; and
acquiring, as the occupant basic state information, information indicating a statistical value of the current state value indicated by the occupant state information acquired by the occupant state acquiring step in a period from start of new traveling of the vehicle until a predetermined period elapses.

40. An awakening level learning method comprising:
acquiring learning model information indicating a learning model that has not been learned or is being learned;
acquiring teacher data used when the learning model indicated by the learning model information acquired by the learning model acquiring step is caused to perform machine learning with supervised learning;
acquiring two or more types of occupant state information different from each other, the occupant state information indicating a current state value which is a state value of an occupant of a vehicle;
acquiring occupant basic state information corresponding to each of the two or more types of occupant state information acquired by the occupant state acquiring step and indicating a basic state value which is the state vale of the occupant when the occupant is in an awakening state;
acquiring difference information indicating a difference between the current state value indicated by the occupant state information acquired by the occupant state acquiring step and the basic state value indicated by the occupant basic state information acquired by the occupant basic state acquiring step and corresponding to the occupant state information, in which two or more types of the difference information corresponding to the two or more types of occupant state information acquired by the occupant state acquiring step are acquired;
generating a learned model for outputting information indicating the awakening level of the occupant as an estimation result by inputting, as explanatory variables, the two or more types of difference information acquired by the difference acquiring step to the learning model indicated by the learning model information acquired by the learning model acquiring step, and causing the learning model to perform machine learning with supervised learning based on the teacher data acquired by the teacher data acquiring step;
outputting the learned model generated by the learning step as learned model information;
identifying the occupant and acquiring personal identification information indicating the identified person; and
acquiring the occupant basic state information corresponding to the personal identification information on a basis of the personal identification information acquired by the occupant identifying step,
wherein the occupant basic state information acquired by the occupant basic state acquiring step is information indicating a statistical value of the current state value indicated by the occupant state information of the occupant indicated by the personal identification information, acquired by the occupant state acquiring step in a period in which the occupant is in the awakening state out of periods in which the occupant was in the vehicle in the past.
